(19) United States
(12) Patent Application Publication
(10) Pub. No.: US 2003/0219455 A1
(43) Pub. Date: Nov. 27, 2003

(54) Glucanosyl transferase-1 protein useful for immunization against Coccidioides spp.

(76) Inventors: Garry T. Cole, Toledo, OH (US); Nelson Delgado, Delran, NJ (US); Jieh-Juen Yu, Toledo, OH (US); Jianmin Xue, Toledo, OH (US)

Correspondence Address: Michael R. Ward, Morrison & Foerster LLP, 425 Market Street, San Francisco, CA, 94105-2482, US

(21) Appl. No.: 10/417,997

(22) Filed: Apr. 16, 2003

Related U.S. Application Data
(60) Provisional application No. 60/374,152, filed on Apr. 22, 2002.

(51) Int. Cl. A61K 39/02; C07H 21/04; C12P 21/04; C07K 14/195; C12N 1/21; C12N 15/74

(52) U.S. Cl. 424/190.1; 435/69.3; 435/70.21; 435/252.3; 435/320.1; 530/350; 536/23.7

(57) ABSTRACT

The present invention provides compositions of 1,3-β-glucanosyltransferase (Gel-1) antigens and polynucleotides encoding the Gel-1 antigens, which Gel-1 antigens are useful for generating an immunological response in an individual and in therapeutic and diagnostic applications of infections due to pathogenic Coccidioides spp. fungi, such as C. immitis or C. posadasii.

### RELATED APPLICATIONS

This application claims benefit of priority under 35 U.S.C. 119(B) of Provisional application No. 60/374,152 filed Apr. 22, 2002, the contents of which are incorporated herein by reference.

### GOVERNMENT LICENSE RIGHTS

The United States Government has a paid up license in this invention and the right in limited circumstances to require the patent owner to license others on reasonable terms as provided for by the terms of Public Service Grants AI 19149, AI37232, and UO1-AI50910 awarded by the National Institutes of Allergy and Infectious Diseases, National Institutes of Health.

### FIELD OF THE INVENTION

The present invention relates generally to the fields of pathogenic fungi and immunology. More particularly, the invention provides compositions of Coccidioides spp. polypeptide antigens and polynucleotides encoding the polypeptides. In particular, this invention provides novel glucanosyl transferase-1 (Gel1) antigens and polynucleotides encoding the Gel1 antigens which are useful for generating or detecting an immunological response and in vaccines and therapeutic applications for infections due to pathogenic Coccidioides spp. fungi, such as C. posadasii or C. immitis.

### BACKGROUND OF THE INVENTION

Coccidioidomycosis, otherwise known as the San Joaquin Valley Fever, is a fungal respiratory disease of humans and wild and domestic animals which is endemic to southwestern United States, northern Mexico, and numerous semiarid areas of Central and South America (Pappagianis, D. Epidemiology of Coccidioidomycosis. Current Topics in Medical Mycology. 1988. 2:199-23). Infection occurs by inhalation of airborne spores (arthroconidia) produced by the saprobic phase of Coccidioides spp. which grows in alkaline desert soil. C. immitis was the first described species, and is now becoming known as the Californian species. The C. posadasii species was recently defined, and was previously recognized as the non-Californian population of C. immitis (Fisher, M. C., Koenig, G. L., White, T. J., Taylor, J. W. Molecular and phenotypic description of Coccidioides posadasii sp. nov., previously recognized as the non-California population of Coccidioides immitis. Mycologia 2002. 94(1):73-84, 2002). The differences in the two species are slight.

It is estimated that 100,000 new cases of this disease occur annually within the rapidly growing population of people who live in regions of the United States between southwest Texas and southern California, where the disease is endemic (Galgiani, J. N. Coccidioidomycosis: A regional disease of national importance; rethinking our approaches to its control. Annals of Internal Medicine. 1999. 130:293-300). Although the majority of immunocompetent individuals are able to resolve their Coccidioides spp. infection spontaneously, the level of morbidity associated even with the primary form of this respiratory mycosis warrants consideration of a vaccine against the disease. Immunocompromised patients, including those infected with human immunodeficiency virus, are at high risk to contract disseminated coccidioidomycosis (Ampel, N. M., C. L. Dols, and J. N. Galgiani. Results of a prospective study in a coccidioidal endemic area. American Journal of Medicine. 1993. 94:235-240). It is also apparent from results of several clinical studies that African-Americans and Asians are genetically predisposed to development of the potentially fatal, disseminated form of the respiratory disease (Galgiani, J. N. 1993. Coccidioidomycosis. Western Journal of Medicine 159:153-171).

The rationale for commitment of research efforts to develop a Coccidioides spp. vaccine is based on clinical evidence that individuals who recover from the respiratory coccidioidomycosis disease retain effective long-term cellular immunity against future infections by the pathogen (Smith, C. E. 1940. American Journal of Public Health 30:600-611). In addition, early preclinical studies demonstrated that a formalin-killed whole-cell (spherule) vaccine prevented deaths in mice after infection with even very large numbers of coccidioidal spores (Levine et al.1961. Journal of Immunology 87:218-227). However, when a similar vaccine preparation was evaluated in a human trial, there was substantial local inflammation, pain, and induration at the injection site, rendering the vaccine unacceptable (Pappagianis et al. Evaluation of the protective efficacy of the killed Coccidioides immitis spherule vaccine in humans. American Review of Respiratory Diseases. 1993. 148:656-660). Further, there was no difference in the number of cases of coccidioidomycosis or the severity of the disease in the formalin-killed spherule vaccinated group compared to the placebo group. Therefore, the original human vaccine trial was not successful.

Subsequent attempts to develop a coccidioidal vaccine focused on crude or partially purified subcellular preparations from the fungus, and had limited success in experimental models (Zimmermann, C. R., S. M. Johnson, G. W. Martens, A. G. White, B. L. Zimmer, and D. Pappagianis. Protection against lethal murine coccidioidomycosis by a soluble vaccine from spherules. Infection and Immunity. 1988. 66:2342-2345; Lecara, G., Cox, R. A., and Simpson, R. B. Coccidioides immitis vaccine: potential of an alkali-soluble, water-soluble cell wall antigen. Infection and Immunity. 1983. 39: 473-475; Cole, G. T., T. N. Kirkland, and S. H. Sun. An immunoreactive, water-soluble conidial wall fraction of Coccidioides immitis 1987. Infection and Immunity 55:657-667; Cole G. T., Kirkland T. N., Franco M., Zhu S., Yuan L., Sun S. H., Hearn V. M. Immunoreactivity of a surface wall fraction produced by spherules of Coccidioides immitis. Infection and Immunity 1988 Oct; 56:2695-701).

In an attempt to discover additional vaccine candidates, we have used a partial genome database derived from C. posadasii for the identification of genes that encode homologs of reported wall-associated proteins. Our rationale for this approach was that some of these proteins may be immunogenic and exposed to the host immune system during the parasitic cycle. It is well-known in the art that one mechanism by which cell wall-associated proteins remain bound to the cell surface is via glycosylphosphatidylinositol (GPI) anchors (de Groot, P. W. J., C. Ruiz, C. R. V. de Aldana, E. Duenas, V. J. Cid, F. D. Ray, J. M. Rodriquez-Pena, P. Perez, A. Andel, J. Caubin, J. Arroyo, J. C. Garcia, C. Gil, M. Molina, L. J. Garcia, C. Nombela, and F. M. Klis. 2001. A genomic approach for the identification and classification of genes involved in cell wall formation and its regulation in Saccharomyces cerevisiae. Comparative and Functional Genomics 2:124-142). GPI-anchored proteins reported in various microbial pathogens have been shown to be immunogenic and suggested to represent important virulence factors (Hung, C.-Y., J.-J. Yu, K. R. Seshan, U. Reichard, and G. T. Cole. 2002. A parasitic phase-specific adhesin of Coccidioides immitis contributes to the virulence of this respiratory fungal pathogen. Infection and Immunity 70:3443-3456; McGwire, B. S., W. A. O'Connell, K.-P. Chang, and D. M. Engman. 2002. Extracellular release of the glycosylphosphatidylinositol (GPI)-linked Leishmania surface metalloproteinase, gp63, is independent of GPI phospholipolysis: implications for parasite virulence. Journal of Biological Chemistry 277:8802-8809). Glucanosyltransferases are representative of such cell wall-bound proteins with GPI-linkages, and have been described in both yeast and filamentous fungi (Bruneau, J.-M., T. Magnin, E. Tagat, R. Legrand, M. Bernard, M. Diaquin, C. Fudali, and J.-P. Latgé. 2001. Proteome analysis of Aspergillus fumigatus identifies glycosylphosphatidylinositol-anchored proteins associated to the cell wall biosynthesis. Electrophoresis 22:2812-2823).

There is a long felt need for a more effective and usable treatment or vaccination regimen to prevent, treat, or ameliorate infection of Coccidioides spp. and disease states associated with the infection.

### SUMMARY OF THE INVENTION

Accordingly, it is an object herein to provide the methods for identifying and isolating polypeptides and nucleic acids encoding polypeptides of Coccidioides spp. that have an immunostimulatory activity. Such immunostimulatory nucleotides and polypeptides will be useful in the prevention, treatment, and diagnosis of infections due to Coccidioides spp.

In order to meet these needs, the present invention provides compositions and methods for the production of glucanosyl transferase-1 (Gel1) polypeptides of Coccidioides spp., including but not limited to the polypeptide sequences of SEQ ID NO:2 and or SEQ ID NO:4.

The present invention also provides polynucleotides encoding the antigens, produced by recombinant technology from the GEL1 gene and gene fragments derived from Coccidioides posadasii, including but not limited to the polynucleotide sequences of SEQ ID NO: 1 and or SEQ ID NO:3.

In one embodiment, the polypeptides of the present invention encompasses the full-length 447 amino acid native protein sequence of SEQ ID NO:2. In another embodiment, the present invention comprises the full length native polypeptide sequence lacking the N-terminal signal peptide amino acids 1 to 18 of SEQ ID NO:2.

In a further embodiment, the polypeptides of the present invention encompasses the full-length 450 amino acid recombinant Gel1 (rGel1) fusion protein sequence of SEQ ID NO:4. In another embodiment, the present invention comprises the full length recombinant polypeptide sequence lacking the N-terminal fusion peptide amino acids 1 to 21 of SEQ ID NO:4.

The present invention also provides the use of the Gel1 polypeptides and polynucleotides encoding the polypeptides of the invention to elicit an immune response sufficient to provide an effective immunization against Coccidioides spp. infection. In one embodiment, the polypeptides provide protection against Coccidioides posadasii and or Coccidioides immitis infections in a mammal, such as a human. In another embodiment, the polypeptides provide protection against Coccidioides spp. infection in domestic animals, including but not limited to dogs, cats, horses, and cattle. In a further embodiment, the invention provides polynucleotides encoding Gel1 polypeptides in a vector suitable for transforming human cells as a method for immunizing humans against Coccidioides spp. infection.

The present invention further provides the compositions and the methods of use of the Gel1 polypeptides in combination with one or more other Coccidioides spp. antigens to elicit an immune response sufficient to provide an effective immunization against Coccidioides spp. infection. In one embodiment the polypeptides are provided as a composition containing a mixture of the polypeptides, for example, Gel1 in combination with Ag2/PRA1-106 (Peng T, Shubitz L, Simons J, Perrill R, Orsborn K I, Galgiani J N. 2002. Localization within Antigen 2/PRA of protective antigenicity for mice against infection with Coccidioides immitis. Infection and Immunity 70(7):3330-3335) and or Coccidioides-immitis specific antigen (referred to hereafter as Csa)(Pan, S. and Cole, G. T. 1995. Molecular and biochemical characterization of Coccidioides immitis-specific (CS) antigen. Infection and Immunity, 63:3994-4002). In another embodiment the composition is provided as a single fusion polypeptide comprised of the Coccidioides spp. polypeptides.

The invention also provides expression vectors that include regulatory sequences such as promoters or other transcriptional regulatory elements operably linked to the nucleotide sequences that control expression of the nucleotide sequences or degenerate variants of the sequences in host cells for the production of the Gel1 polypeptides of SEQ ID NO:2 and or SEQ ID NO:4.

The invention further provides host cells derived from yeast, bacterial, plant, animal or human sources containing the expression vectors comprising the sequences of SEQ ID NO: 1. Additionally, the invention provides host cells derived from yeast, bacterial, plant, animal or human sources containing the expression vectors comprising the sequences of SEQ ID NO:3.

The present invention also provides shorter polypeptide fragments included within amino acids 1 to 447 of SEQ ID NO:2 and or amino acids 1 to 450 of SEQ ID NO:4. In preferred embodiments, these shorter polypeptides may include polypeptides of not less than 25 amino acids in length, inclusive of 26, 27, 28, 29, 30, 31, 32, 33, 34, 35, 36, 37, 38, 39, 40, 41, 42, 43, 44,45,46,47,48,49,50,51,52,53,54,55,56,57,58,59,60,61,62,63,64,65,66,67,68,69, 70, 71, 72, 73, 74, 75, 76, 77, and 78-150 amino acids in length. Such polypeptide fragments, which are substantially the same amino acid length as the sequences containing the antigenic epitopes, provide similar ability to elicit an immune response, including such immune responses that provides protection against Coccidioides spp. infection.

In another embodiment, the present invention includes polypeptides that are substantially identical to the polypeptide sequences of SEQ ID NO:2 and or SEQ ID NO:4 and or contain at least one conservative amino acid substitution. Such polypeptides, which are substantially the same amino acid length, provide similar ability to elicit an immune response, including such immune responses that provides protection against Coccidioides spp. infection. Such polypeptides, having substantial identity to the polypeptide sequences of Gel1 and or rGel1, include those polypeptides at least about 99% identical or equivalent, at least about 95% identical or equivalent, at least about 90% identical or equivalent, at least about 85% identical or equivalent, at least about 80% identical or equivalent, at least about 75% identical or equivalent, and at least about 70% identical to said polypeptides and which have the aforementioned activities, are encompassed in the invention.

The present invention further provides methods and compositions of isolated polypeptides identical or substantially identical to the polypeptide sequences of SEQ ID NO:2 and or SEQ ID NO: 4 useful in pharmaceutical compositions.

The present invention also provides vaccine formulations and methods of preparing the formulations containing the Gel1 polypeptides and or polynucleotides encoding the polypeptides. The present invention further provides vaccine formulations containing adjuvants and pharmaceutical excipients and carriers.

The present invention provides the Gel1 vaccine formulations and methods for eliciting an effective immune response in a mammal, including humans and domestic animals, for the prevention of Coccidioides spp. infections.

The present invention further provides kits containing the Gel1 polypeptides and or other Coccidioides spp. antigens and or polynucleotides encoding the polypeptides, to facilitate the use of the polypeptides and or polynucleotides.

The present invention also provides a construct comprising a promoter sequence for the Gel1 gene encoding the polypeptides, which can direct gene expression in a host cell. The present invention also provides the use of the Gel1 polypeptides and or polynucleotides encoding the Gel1 polypeptides in diagnostic kits for the detection of infections due to Coccidioides spp. in mammals, such as humans and domestic animals.

The present invention also provides an antibody specific for an antigen of the Gel1 polypeptide and methods for the creation of such antibodies. Such antibodies may be used in diagnostic kits for the detection of infections due to Coccidioides spp. The present invention provides kits containing antibodies in suitable compositions for the detection of infections due to Coccidioides spp. in mammals, such as humans and domestic animals.

The above and other aspects of the invention will become readily apparent to those of skill in the art from the following detailed description and figures, wherein only the preferred embodiments of the invention are shown and described, simply by way of illustration of the best mode of carrying out the invention. As is readily recognized, the invention is capable of modifications within the skill of the relevant art without departing from the spirit and scope of the invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B depict nucleotide (SEQ ID NO:1) and deduced amino acid sequences (SEQ ID NO:2) of the C. posadasii GEL1 gene [amino acids are displayed in a single-letter abbreviation after alignment for maximal identity by the program CLUSTAL X, version 1.8)]. The ORF of GEL-1 deduced from the genomic sequence is interrupted by 6 introns. The translated amino acid sequence shows a protein of 447 residues with a predicted molecular weight of 48.1 kDa. The hydrophobic signal peptide, consisting of 18 amino acids, is located at the N-terminal region. There is a GPI-anchor site at amino acid number 413 (i.e., G⁴¹³)..

FIGS. 2A and 2B depict the aligned nucleotide (SEQ ID NO:3) and deduced amino acid sequences (SEQ ID NO:4) of the rGEL1 gene construct expressed in an E. coli host. The differences from the genomic sequence include a lack of the nucleotides encoding the 18 amino acids of the N-terminal signal peptide of the native Gel1 polypeptide and the inclusion of 63 nucleotides in the recombinant construct encoding a 21 amino acid fusion peptide on the N-terminal derived from the pET28b vector. Additionally, the recombinant construct contains differences in 3 nucleotides in the encoding region compared to the genomic sequence; i.e., nucleotide nos. 446 (resulting in a G at amino acid no. 149), 473 (resulting in a P at amino acid no. 158), and 1278 (resulting in no change in amino acid). The translated amino acid sequence shows a protein of 450 residues with a predicted molecular weight of 48 kDa, which includes the vector-encoded fusion partner peptide.

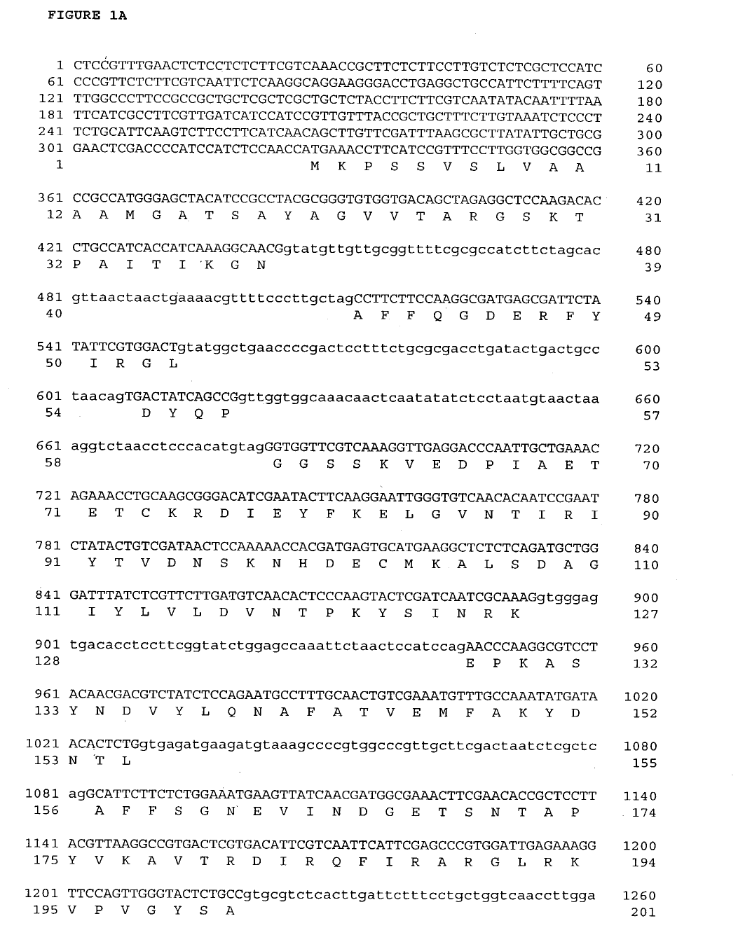

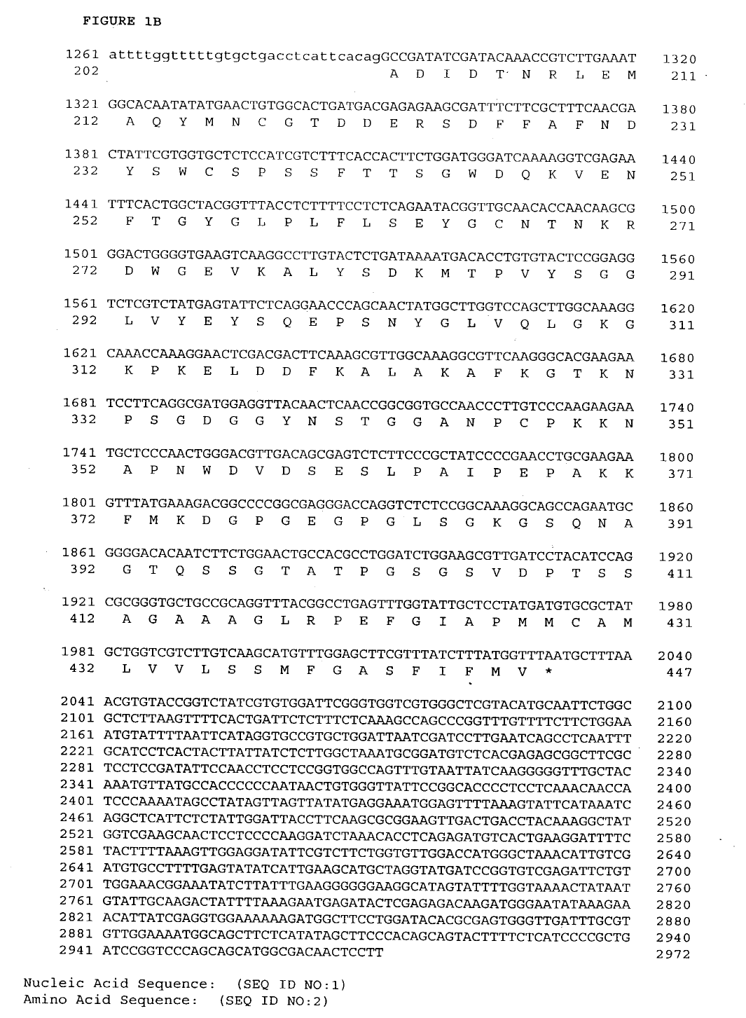

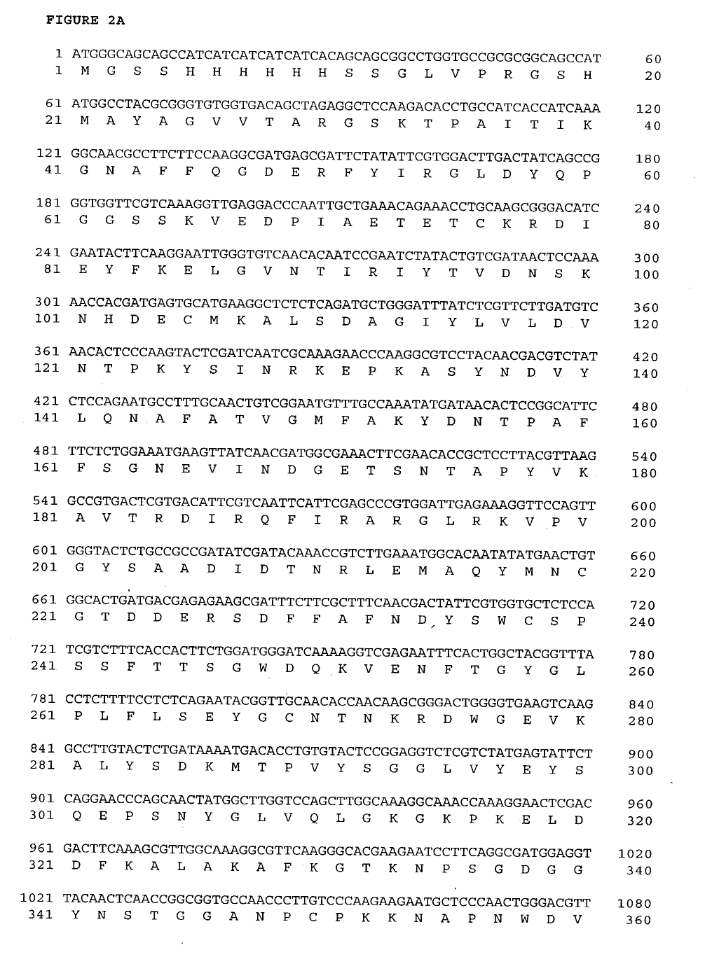

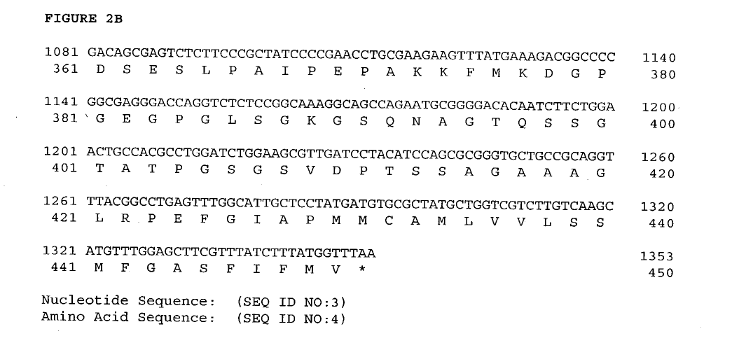

### BRIEF DESCRIPTION OF THE SEQUENCE LISTINGS

SEQ ID NO: 1 depicts the determined nucleotide sequence encoding genomic GEL1, including the terminal signal sequence;

SEQ ID NO:2 depicts the deduced amino acid sequence of the native Gel1 polypeptide encoded by the nucleotide sequence of SEQ ID NO: 1, including the 18 amino acid sequence of the N-terminal signal peptide;

SEQ ID NO:3 depicts the determined nucleotide sequence of the recombinant construct containing GEL1 CDNA and vector construct sequences that encode a fusion polypeptide. In the present sequence, nucleotides 1 through 63 are derived from the pET28b vector and encode an affinity tag, while nucleotides 64 through 1353 are derived from GEL1 cDNA;

SEQ ID NO:4 depicts the deduced amino acid sequence of the recombinant Gel1 fusion polypeptide encoded by the nucleotide sequence of SEQ ID NO:3, including 21 N-terminal amino acids derived from the pET28b vector;

SEQ ID NO:5 depicts the nucleotide sequence of the sense primer used for subcloning the GEL1 sequence into the pET28b vector at the NdeI restriction site;

SEQ ID NO:6 depicts the nucleotide sequence of the antisense primer used for subcloning the GEL1 sequence into the pET28b vector at the SalI restriction site;

SEQ ID NO:7 depicts the nucleotide sequence of the sense primer used to amplify by PCR a partial (391 bp) fragment of the C. posadasii GEL1 gene, using genomic DNA as template. The PCR product was cloned into the pCR2.1 TOPO cloning vector;

SEQ ID NO:8 depicts the nucleotide sequence of the antisense primer used to amplify by PCR a partial (391 bp) fragment of the C. posadasii GEL1 gene, using genomic DNA as template. The PCR product was cloned into the pCR2.1 TOPO cloning vector;

SEQ ID NO:9 depicts the nucleotide sequence of a Gene-Specific Primer (GSP) used for a primary amplification of the C. posadasii GEL1 genomic sequence using genome walking, which is a PCR-based method used for sequencing genomic DNA upstream or downstream of a partial gene sequence. This GSP was used in combination with an adapter primer (AP-1) provided in the GenomeWalker™ Kit (Clontech);

SEQ ID NO:10 depicts the nucleotide sequence of the AP-1 primer used in genome walking;

SEQ ID NO: 11 depicts the nucleotide sequence of a nested Gene-Specific Primer (GSP) used for a secondary amplification of the C. posadasii GEL1 genomic sequence using genome walking. This GSP was used in combination with a nested adapter primer (AP-2) provided in the GenomeWalker™ Kit (Clontech);

SEQ ID NO: 12 depicts the nucleotide sequence of the AP-2 primer used in genome walking;

SEQ ID NO: 13 depicts the nucleotide sequence of the GEL1 specific sense primer used to PCR then clone the full-length genomic sequence into the pCR2.1 TOPO vector;

SEQ ID NO: 14 depicts the nucleotide sequence of the GEL1 specific antisense primer used to PCR then clone the full-length genomic sequence into the pCR2.1 TOPO vector;

SEQ ID NO: 15 depicts the nucleotide sequence of the synthetic CpG adjuvant used in animal experiments;

SEQ ID NO: 16 depicts the amino acid sequence of a fusion partner peptide at the N-terminal of rGel1; and

SEQ ID NO: 17 depicts the N-terminal amino acid sequence of the 48-kDa or Gel1.

### DETAILED DESCRIPTION OF THE INVENTION

Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Reference is made to standard textbooks of molecular biology that contain definitions and methods and means for carrying out basic techniques, encompassed by the present invention. See, for example, Sambrook et al., Molecular Cloning: A Laboratory Manual, Third Edition, Cold Spring Harbor Laboratory Press, New York (2001), Current Protocols in Molecular Biology, Ausubel et al (eds.), John Wiley & Sons, New York (2001) and the various references cited therein.

I. The Polypeptide Sequences of the Invention.

The invention focuses on the use of glucanosyltransferase-1 (Gel1) polypeptides and the nucleotide sequences that encode them as immunogenic antigens for a preventative or therapeutic vaccine for coccidioidomycosis, or for detection of immune responses in individuals infected by Coccidioides spp.

The Gel1 of the present invention encodes a glycosylphosphatidylinositol (GPI)-anchored protein that shares homology with several yeast proteins such as Gas1p of S. cerevisiae (Nuoffer, C., Jeno, P., Conzelmann, A., Horvath, A., and Riezman, H. (1991) Molecular and Cellular Biology 11, 27-37; Popolo, L., Vai, M., Gatti, E., Porello, S., Bonfante, P., Balestrini, R., and Alberghina, L. (1993) Journal of Bacteriology 175, 1879-1885; Ram, A. F. J., Brekelmans, S. S. C., Oehlen, L. J. W. M., and Klis, F. M. (1995) FEBS Lett. 358, 165-170; Vai, M., Gatti, E., Lacana, E., Popolo, L., and Alberghina, L. (1991) J. Biol. Chem. 266, 12242-12248) or Phrp of Candida albicans (Muhlschlegel, F. A., and Fonzi, W. A. (1997) Mol. Cell. Biol. 17, 5960-5967; Saporito-Irwin, S. M., Birse, C. E., Sypherd, P. S., and Fonzi, W. A. (1995) Molecular and Cellular Biology 15, 601-613). GAS1, PHR1, and PHR2 gene products are required for correct morphogenesis in yeast and were so far endowed with an unknown biochemical function. Further Gas1p, Phr1p, and Phr2p display the same 1,3-β-glucanosyltransferase activity as Gel1 of A. fumigatus.

Native Gel1 from Coccidioides spp. is a protein of 447 amino acids that contains an 18 amino acid signal peptide with a predicted cleavage site at S¹⁸/A¹⁹ and a predicted a glycosylphosphatidylinositol (GPI) anchor site at G⁴¹³. The amino acid sequence of native Gel1 is shown in SEQ ID NO:2. The rGel1 is a protein of 450 amino acids; the differences between the native Gel1 and rGel1 is the lack of the 18 amino acid signal peptide and inclusion of a 21 amino acid fusion partner peptide at the N-terminal of the rGel1 (MGSSHHHHHHSSGLVPRGSHM) (SEQ ID NO: 16) derived from the pET-28b vector. Additionally, the recombinant construct contains differences in 3 nucleotides in the encoding region compared to the genomic sequence; i.e., nucleotide nos. 446 (resulting in a G at amino acid no. 149), 473 (resulting in a P at amino acid no. 158), and 1278 (resulting in no change in amino acid). The amino acid sequence of rGel1 is shown in SEQ ID NO:4.

Additional polypeptides are encompassed in the invention, consisting essentially of the sequences of the Gel1 and or rGel1 polypeptides, including polypeptides at least about 99% identical or equivalent, at least about 95% identical or equivalent, at least about 90% identical or equivalent, at least about 85% identical or equivalent, at least about 80% identical or equivalent, at least about 75% identical or equivalent, and at least about 70% identical to the sequences of the polypeptides.

As used herein, the terms “protein” or “polypeptide” are used in the broadest sense to mean a sequence of amino acids that can be encoded by a cellular gene or by a recombinant nucleic acid sequence or can be chemically synthesized. A protein can be a complete, full length gene product, which can be a core protein having no amino acid modifications, or can be a post-translationally modified form of a protein such as a phosphoprotein, glycoprotein, proteoglycan, lipoprotein or nucleoprotein. In some cases, the term “polypeptide” is used in referring to a portion of an amino acid sequence (peptides) of a full length protein. An active fragment of a Gel1 is an example of such a polypeptide. The reasons for reducing the full-length antigen to fragments are multiple; 1) a positive correlation between development of DTH (delayed-type hypersensitivity) (a Th1 immune response) to coccidioidal antigens and the ability to resist disseminated coccidioidomycosis has been shown. This response is generally regarded as a MHC II type response (Louie et al. 1999. Influence of host genetics on the severity of coccidioidomycosis. Emerging Infectious Diseases 5:672-680) and that peptides that bind to MHC II receptors are generally 13-25 residues in length; 2) determination of epitopes within a larger polypeptide or multiple peptides that enhance or suppress an immune response allow for the creation of fusion proteins containing select epitopes that can be used as vaccines with increased potency (Sette A, et al. 2002. Optimizing vaccine design for cellular processing, MHC binding and TCR recognition. Tissue Antigens 59:443-451); and 3) reducing the size of the polypeptide can lead to important advantages in the production, purification and safety of a vaccine.

“Consisting essentially of”, in relation to amino acid sequence of a polypeptide, protein or peptide, is a term used hereinafter for the purposes of the specification and claims to refer to a conservative substitution or modification of one or more amino acids in that sequence such that the tertiary configuration of the polypeptide, protein or peptide is substantially unchanged. Polypeptides, consisting essentially of the sequence of the polypeptides of Gel1 and or rGel1, include those polypeptides at least about 99% identical or equivalent, at least about 95% identical or equivalent, at least about 90% identical or equivalent, at least about 85% identical or equivalent, at least about 80% identical or equivalent, at least about 75% identical or equivalent, and at least about 70% identical to the sequences of the polypeptides are encompassed in the invention.

“Conservative substitutions” is defined by substitutions of amino acids having substantially the same charge, size, hydrophilicity, and or aromaticity as the amino acid replaced. Such substitutions, known to those of ordinary skill in the art, include glycine-alanine-valine; isoleucine-leucine; tryptophan-tyrosine; aspartic acid-glutamic acid; arginine-lysine; asparagine-glutamine; and serine-threonine.

“Modification”, in relation to amino acid sequence of a polypeptide, protein or peptide, is defined functionally as a deletion of one or more amino acids which does not impart a change in the conformation, and hence the biological activity, of the polypeptide, protein or peptide sequence.

The common amino acids are generally known in the art. Additional amino acids that may be included and or substituted in the peptide of the present invention include: L-norleucine; aminobutyric acid; L-homophenylalanine; L-norvaline; D-alanine; D-cysteine; D-aspartic acid; D-glutamic acid; D-phenylalanine; D-histidine; D-isoleucine; D-lysine; D-leucine; D-methionine; D-asparagine; D-proline; D-glutamine; D-arginine; D-serine; D-threonine; D-valine; D-tryptophan; D-tyrosine; D-omithine; aminoisobutyric acid; L-ethylglycine; L-t-butylglycine; penicillamine; I-naphthylalanine; cyclohexylalanine; cyclopentylalanine; aminocyclopropane carboxylate; aminonorbornylcarboxylate; L-α-methylalanine; L-α-methylcysteine; L-α-methylaspartic acid; L-α-methylglutamic acid; L-α-methylphenylalanine; L α-methylhistidine; L-α-methylisoleucine; L-α-methyllysine; L-α-methylleucine; L-α-methylmethionine; L-α-methylasparagine; L-α-methylproline; L-α-methylglutamine; L-α-methylarginine; L-α-methylserine; L-α-methylthreonine; L-α-methylvaline; L-α-methyltryptophan; L-α-methyltyrosine; L-α-methylomithine; L-α-methylnorleucine; amino-α-methylbutyric acid;. L-α-methylnorvaiine; L-α-methylhomophenylalanine; L-α-methylethylglycine; methyl-γ-aminobutyric acid;. methylaminoisobutyric acid; L-α-methyl-t-butylglycine; methylpenicillamine; methyl-α-naphthylalanine; methylcyclohexylalanine; methylcyclopentylalanine; D-α-methylalanine; D-α-methylornithine; D-α-methylcysteine; D-α-methylaspartic acid; D-α-methylglutamic acid; D-α-methylphenylalanine; D-α-methylhistidine; D-α-methylisoleucine; D-α-methyllysine; D-α-methylleucine; D-α-methylmethionine; D-α-methylasparagine; D-α-methylproline; D-α-methylglutamine; D-α-methylarginine; D-α-methylserine; D-α-methylthreonine; D-α-methylvaline; D-α-methyltryptophan; D-α-methyltyrosine; L-N-methylalanine; L-N-methylcysteine; L-N-methylaspartic acid; L-N-methylglutamic acid; L-N-methylphenylalanine; L-N-methylhistidine; L-N-methylisoleucine; L-N-methyllysine; L-N-methylleucine; L-N-methylmethionine; L-N-methylasparagine; N-methylcyclohexylalanine; L-N-methylglutamine; L-N-methylarginine; L-N-methylserine; L-N-methylthreonine; L-N-methylvaline; L-N-methyltryptophan; L-N-methyltyrosine; L-N-methylomithine; L-N-methylnorleucine; N-amino-α-methylbutyric acid; L-N-methylnorvaline; L-N-methylhomophenylalanine; L-N-methylethylglycine; N-methyl-γaminobutyric acid; N-methylcyclopentylalanine; L-N-methyl-t-butylglycine; N-methylpenicillamine; N-methyl-α-naphthylalanine; N-methylaminoisobutyric acid; N-(2-aminoethyl)glycine; D-N-methylalanine; D-N-methylomithine; D-N-methylcysteine; D-N-methylaspartic acid; D-N-methylglutamic acid; D-N-methylphenylalanine; D-N-methylhistidine; D-N-methylisoleucine; D-N-methyllysine; D-N-methylleucine; D-N-methylmethionine; D-N-methylasparagine; D-N-methylproline; D-N-methylglutamine; D-N-methylarginine; D-N-methylserine; D-N-methylthreonine; D-N-methylvaline; D-N-methyltryptophan; D-N-methyltyrosine; N-methylglycine; N-(carboxymethyl)glycine; N-(2-carboxyethyl)glycine; N-benzylglycine; N-(imidazolylethyl)glycine; N-(1-methylpropyl)glycine; N-(4-aminobutyl)glycine; N-(2-methylpropyl)glycine; N-(2-methylthioethyl)glycine; N-(hydroxyethyl)glycine; N-(carbamylmethyl)glycine; N-(2-carbamylethyl)glycine; N-(1-methylethyl)glycine; N-(3-guanidinopropyl)glycine; N-(3-indolylethyl)glycine; N-(p-hydroxyphenethyl)glycine; N-(1-hydroxyethyl)glycine; N-(thiomethyl)glycine; N-(3-aminopropyl)glycine; N-cyclopropylglycine; N-cyclobutyglycine; N-cyclohexylglycine; N-cycloheptylglycine; N-cyclooctylglycine; N-cyclodecylglycine; N-cycloundecylglycine; N-cyclododecylglycine; N-(2,2-diphenylethyl)glycine; N-(3,3-diphenylpropyl)glycine; N-(N-(2,2-diphenylethyl)carbamylmethyl)glycine; N-(N-(3,3-diphenylpropyl)carbamylmethyl)glycine; and 1-carboxy-1-(2,2-diphenylethylamino)cyclopropane.

The polypeptides of the present invention can be produced by a known chemical synthesis method; for example, a liquid phase synthesis method, a solid phase synthesis method, and others (Izumiya, N., Kato, T., Aoyagi, H., Waki, M., “Basis and Experiments of Peptide Synthesis”, 1985, Maruzen Co., Ltd.).

The polypeptides of the present invention may contain one or more protected amino acid residues. The protected amino acid is an amino acid whose functional group or groups is/are protected with a protecting group or groups by a known method or by the use of various protected amino acids that are commercially available.

II. The DNA Sequences of the Invention.

Alternatively, the polypeptides of the present invention can be produced by producing a polynucleotide (DNA or RNA) which encodes the amino acid sequence of a polypeptide of the present invention and producing said polypeptide by a genetic engineering technique using the polynucleotide. Polynucleotide coding sequences for amino acid residues are known in the art and are disclosed for example in Molecular Cloning: A Laboratory Manual, Third Edition, Sambrook, Fritsch, and Maniatis, Cold Spring Harbor Laboratory Press, 2001.

The genomic GEL1 sequence of the present invention is comprised of 2972 nucleotide residues that encodes a polypeptide with 447 amino acids. The ORF of GEL1 deduced from the genomic sequence is interrupted by 6 introns. The genomic sequence encoding Gel1 is shown in SEQ ID NO: 1. The nucleotide sequence encoding the rGel1 of the present invention has 1353 nucleotide residues, including a stop codon, and results in a recombinant polypeptide with 450 amino acids. The nucleotide sequence of the rGel1 construct is shown in SEQ ID NO:3, which includes 63 nucleotides derived from the pET28b vector.

Within the context of the present invention “polynucleotide” in general relates to polyribonucleotides and polydeoxyribonucleotides, it being possible for these to be non-modified RNA or DNA or modified RNA or DNA. Polynucleotides which encode the peptides of the present invention mean the sequences exemplified in this application as well as those which have substantial identity to those sequences and which encode the peptides. Preferably, such polynucleotides are those which hybridize under stringent conditions as defined herein and are at least 70%, preferably at least 80% and more preferably at least 90% to 95% identical to those sequences.

“Consisting essentially of”, in relation to a nucleic acid sequence, is a term used hereinafter for the purposes of the specification and claims to refer to sequences of the present invention and sequences with substitution of nucleotides as related to third base degeneracy. As appreciated by those skilled in the art, because of third base degeneracy, almost every amino acid can be represented by more than one triplet codon in a coding nucleotide sequence. Further, minor base pair changes may result in variation (conservative substitution) in the amino acid sequence encoded, and are not expected to substantially alter the biological activity of the gene product. Thus, a nucleic acid sequencing encoding a protein or peptide as disclosed herein, may be modified slightly in sequence (e.g., substitution of a nucleotide in a triplet codon), and yet still encode its respective gene product of the same amino acid sequence.

The terms “stringent conditions” or “stringent hybridization conditions” includes reference to conditions under which a polynucleotide will hybridize to its target sequence, to a detectably greater degree than other sequences (e.g., at least 2-fold over background). In particular, a DNA or polynucleotide molecule which hybridizes under stringent conditions is preferably at least 70%, 75%, 80%, 85%, 90%, 95%, 97%, 98% or 99% identical to the DNA that encodes the amino acid sequences described herein. In a preferred embodiment these polynucleotides that hybridize under stringent conditions also encode a protein or peptide which upon administration to a subject provides an immunostimulation sufficient to provide some level of immune protection against Coccidioides spp. infection as described herein.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short polynucleotides (e.g., 10 to 50 nucleotides) and at least about 60° C. for long polynucleotides (e.g., greater than 50 nucleotides)—for example, “stringent conditions” can include hybridization in 50% formamide, 1 M NaCl, 1% SDS at 37° C., and three washes, 15 minutes each, in 0.1×SSC at 60 to 65° C.

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman (Advances in Applied Mathematics. 1981. 2: 482-489), to find the best segment of identity or similarity between two sequences. Gap performs global alignments: all of one sequence with all of another similar sequence using the method of Needleman and Wunsch, (Journal of Molecular Biology. 1970. 48:443-453). When using a sequence alignment program such as BestFit to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Naturally, the present invention also encompasses DNA segments that consist essentially of or are complementary, or essentially complementary, to the sequence set forth in SEQ ID NO:1 and SEQ ID NO:3. Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the nucleic acid segment SEQ ID NO:1 or SEQ ID NO:3 under stringent conditions such as those described herein.

The nucleic acid segments of the present invention, regardless of the length of the coding sequence itself, may be combined with other nucleic acid and DNA sequences, such as promoters, polyadenylation signals, additional restriction enzyme sites, multiple cloning sites, other coding segments, and the like, such that their overall length may vary considerably. It is therefore contemplated that a nucleic acid segment or fragment of almost any length may be employed, with the total length preferably being limited by the ease of preparation and use in the intended recombinant protocol.

For example, nucleic acid segments or fragments may be prepared that include a short contiguous stretch identical to or complementary to SEQ ID NO:1 or SEQ ID NO:3, such as about a 15, 18 or 21 nucleotide stretch, up to about 20,000, about 10,000, about 5,000 or about 3,000 base pairs in length. Nucleic acid and DNA segments with total lengths of about 1,000, about 500, about 200, about 100 and about 50 base pairs in length (including all intermediate lengths) are also contemplated to be useful.

It will be readily understood that “intermediate lengths”, in these contexts, means any length between the quoted ranges, such as 21, 22, 23, 24, 25, etc.; 30, 31, 32, etc.; 50, 51, 52, 53, etc.; 100, 101, 102, 103, etc.; 150, 151, 152, 153, etc.; including all integers through the 200-500; 500-1,000; 1,000-2,000; 2,000-3,000; 3,000-5,000; 5,000-10,000 ranges, up to and including sequences of about 12,001, 12,002, 13,001, 13,002, 15,001, 20,001 and the like.

It will also be understood that this invention is not limited to the particular nucleic acid and amino acid sequences of SEQ ID NO:1, SEQ ID NO:2, SEQ ID NO:3 and SEQ ID NO:4, respectively. Recombinant vectors and isolated DNA segments may therefore variously include the coding region from SEQ ID NO:1 or SEQ ID NO:3, coding regions bearing selected alterations or modifications in the basic coding region, or they may encode larger polypeptides that nevertheless include such coding regions or may encode biologically functional equivalent proteins or peptides that have variant amino acids sequences.

The nucleic acid and DNA segments of the present invention further include sequences that encode biologically functional equivalent Coccidioides spp. peptides that arise as a consequence of codon redundancy and functional equivalency that are known to occur naturally within nucleic acid sequences and the proteins thus encoded. Equally, functionally equivalent proteins or peptides may be created via the application of recombinant DNA technology in which changes in the protein structure may be engineered, based on considerations of the properties of the amino acids being exchanged. Changes designed by human intervention may be introduced through the application of site-directed mutagenesis techniques, e.g., to introduce improvements to the antigenicity of the protein.

III. Expression Vectors, Hosts, and Expression of Polypeptides of the Invention in vitro and in vivo.

The term “expression vector” refers to a polynucleotide that includes coding sequences that encode the polypeptide of the invention and provides the sequences necessary for its expression in the selected host cell. Expression vectors will generally include a transcriptional promoter and terminator, or will provide for incorporation adjacent to an endogenous promoter. Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

The recombinant host cells of the present invention may be maintained in vitro, e.g., for recombinant protein, polypeptide or peptide production. Equally, the recombinant host cells could be host cells in vivo, such as results from immunization of an animal or human with a nucleic acid segment of the invention. Accordingly, the recombinant host cells may be prokaryotic or eukaryotic host cells, such as E. coli, Saccharomyces cerevisiae or other yeast, mammalian or human or plant host cells. It will be further appreciated by the skilled practitioner that other prokaryotic and eukaryotic cells and cell lines may be appropriate for a variety of purposes; e.g., to provide higher expression, desirable glycosylation patterns, or other features. Expression vectors will usually be plasmids, further comprising an origin of replication and one or more selectable markers. The pET28b-GEL1 constuct of the present invention is an example of such expression vectors. A YEpFLAG-1-GEL1 construct is another example. However, expression vectors may alternatively be viral recombinants designed to infect the host, or integrating vectors designed to integrate at a preferred site within the host's genome. Examples of other expression vectors are disclosed in Molecular Cloning: A Laboratory Manual, Third Edition, Sambrook, Fritsch, and Maniatis, C old Spring Harbor Laboratory Press, 2001.

Such polynucleotides encoding the polypeptides of the invention and expression vectors carrying the vectors can be used to produce the polypeptides in vitro or in vivo. The polypeptides so produced can be isolated according to the procedures described herein and commonly known in the art and then used in a therapeutic or immunization protocol.

One may also prepare fusion proteins and peptides, e.g., where the Cocciclioicles spp. peptide coding region is included within the same expression unit with other proteins or peptides having desired functions, such as for purification or immunodetection purposes (e.g., proteins that may be isolated by affinity chromatography and enzyme label coding regions, respectively), or proteins and peptides encoding additional antigens capable of eliciting an immunostimulatory response in a subject (e.g., such as the Coccidioides spp. polypeptides Ag2/Pra, Ure, Csa, or non-Coccidioides protein antigens or toxoids, such as tetanus toxoid, diphtheria toxoid, cholera toxoid, ovalbumin, or keyhole limpet haemocyanin).

In another embodiment, the present invention provides polynucleotide based vaccines or immune-stimulatory formulations, whereby the polynucleotide(s) encoding the polypeptides are administered directly to the subject patient in need thereof, provided the polynucleotide has the appropriate transcriptional control regions to direct the expression of the coding sequence contained in the polynucleotide or expression vector.

Therefore, the present invention also provides DNA based vaccines or immunogenic compositions to provide one or more of the polypeptides described herein. DNA vaccines have been developed for a number of diseases, whereby a DNA vaccine contains a DNA encoding an antigen cloned in a plasmid vector. It will be apparent to one skilled in the art that the immunostimulatory activity of the polypeptides encoded by the DNA sequences disclosed herein lies not in the precise nucleotide sequence of the DNA sequences, but rather in the epitopes inherent in the amino acid sequences encoded by the DNA sequences. It will therefore also be apparent that it is possible to recreate the immunostimulatory activity of one of these polypeptides by recreating the epitope, without necessarily recreating the exact DNA sequence. Such sequences may differ by reason of the redundancy of the genetic code from the sequences disclosed herein. Accordingly, the degeneracy of the genetic code further widens the scope of the present invention as it enables major variations in the nucleotide sequence of a DNA molecule while maintaining the amino acid sequence of the encoded protein or a protein that consists essentially of the same sequence. Such degeneracy is described in U.S. Pat. No. 6,228,371, the contents of which are incorporated herein by reference.

The expression of the antigen or polypeptide may be improved by providing a strong promoter such as, for example, Rous Sarcoma Virus LTR, the cytomegalovirus immediate early promoter, and the SV40 T antigen promoter. Success with DNA vaccines has been demonstrated using a variety of antigens for a number of diseases (U.S. Pat. Nos. 6,384,018, 6,284,533, 6,165,993, the contents of which are incorporated herein by reference). The DNA vaccine or immune stimulating composition may further include an acceptable carrier or liposome as described in U.S. Pat. No. 5, 703,055, which is incorporated herein by reference; and can be made in accordance with known methods as described in, for example, U.S. Pat. Nos. 5,589,466; 5,580,859; 5,561,064; and 6,339,068, the contents of which are incorporated herein by reference.

The delivery of the DNA vaccine or immunostimulatory composition can be accomplished using a variety of procedures commonly employed in the art. For non-viral DNA transfer in cultured cells, examples of such methods include calcium phosphate mediated, DEAE-dextran, electroporation, direct microinjection, liposome mediated delivery, cell sonication, and receptor mediated gene targeting which utilize a cell-receptor-specific ligand and an DNA binding agent, which mediate the uptake of a gene into a specific cell type based on the interaction of the ligand and the receptor. The recombinant DNA encoding the polypeptides of the present invention can also be provided to the cells by direct injection of the naked DNA or plasmid DNA or coupled to particle bombardment with known methods as described in, for example, U.S. Pat. No. 5,865,796, incorporated herein by reference.

In another embodied method of delivering the DNA vaccine or immunostimulatory composition to the cell, viral-vector mediated delivery can be used. Examples of viral vectors for such delivery include adeno-associated virus (AAV) (U.S. Pat. No. 5,843,742 incorporated herein by reference), adenovirus (U.S. Pat. Nos. 6,410,010 and 6,403,370, incorporated herein by reference), vaccinia virus (U.S. Pat. Nos. 6,287,570, and 6,214,353 incorporated herein by reference), herpesvirus, canarypox virus (U.S. Pat. No. 6,183,750 incorporated herein by reference), other Poxviruses, Retrovirus, and other RNA or DNA viral expression vectors known in the art.

The vectors used to deliver the polypeptides of the present invention may be maintained as an episome or stably integrated into the chromosome of the cell.

IV. How the Polypeptide May Be Isolated.

The peptides and polypeptides of the present invention, when produced, can be purified by isolation and purification methods for proteins generally known in the field of protein chemistry. Within the context of the present invention, “isolated” means separated out of its natural environment. An “isolated polypeptide” is, in this context, a substantially pure polypeptide.

The term “substantially pure polypeptide” means a polypeptide that has been separated from at least some of those components which naturally accompany it, such as other contaminating polypeptides, polynucleotides, and or other biological materials often found in cell extracts. Typically, the protein is substantially pure when it is at least 60%, by weight, free from the proteins and other naturally-occurring organic molecules with which it is naturally associated in vivo. Preferably, the purity of the preparation is at least 75%, more preferably at least 90%, and most preferably at least 99%, by weight. A substantially pure Gel1 polypeptide may be obtained, for example, by extraction from a natural source, or by expression of a recombinant nucleic acid encoding an immunoreactive Gel1 polypeptide, such as the nucleic acid molecule shown as SEQ ID NO: 3, using methods described herein. In addition, an amino acid sequence consisting of at least an immunogenic portion of the amino acid sequence of SEQ ID NO: 4 can be chemically synthesized in a substantially pure form.

Methods of purification include, for example, extraction, recrystallization, ammonium sulfate precipitation, sodium sulfate, centrifugation, dialysis, ultrafiltration, adsorption chromatography, ion exchange chromatography, hydrophobic chromatography, normal phase chromatography, reversed-phase chromatography, gel filtration method, gel permeation chromatography, affinity chromatography, electrophoresis, countercurrent distribution, combinations of these, and other know protein or peptide purification methods are well known to those of skill in the art and can be used herein.

Purity can be measured by any appropriate method, e.g., HPLC analysis, immunoaffinity chromatography using an antibody specific for the Gel1 polypeptide, polyacrylamide gel electrophoresis, and the like.

A Coccidioides spp. polypeptide that is “isolated to homogeneity,” as applied to the present invention, means that the Coccidioides spp. polypeptide has a level of purity where the Coccidioides spp. polypeptide is substantially free from other proteins, peptides and biological components. For example, a isolated Coccidioides spp. polypeptide will often be sufficiently free of other peptide and protein components so that sequencing may be performed successfully or that pharmaceutically acceptable formulations can be created. However, this does not exclude the re-mixing of the peptides of the invention, once isolated, with other vaccine components.

V. Preparation and Formulation of Vaccines.

The polypeptides and formulations employing the polypeptides may also be in the form of a peptide salt thereof. In view of the utility of the polypeptides of the present invention, preferred salts include those salts that are pharmaceutically acceptable for administration into a subject patient.

The polypeptides of the present invention may form a salt by addition of an acid. Examples of the acid include inorganic acids (such as hydrochloric acid, hydrobromic acid, phosphoric acid, nitric acid, and sulfuric acid) or organic carboxylic acids (such as acetic acid, propionic acid, maleic acid, succinic acid, malic acid, citric acid, tartaric acid, and salicylic acid), acidic sugars such as glucuronic acid, galacturonic acid, gluconic acid, ascorbic acid, etc., acidic polysaccharides such as hyaluronic acid, chondroitin sulfates, alginic acid, or organic sulfonic acids (such as methanesulfonic acid, and p-toluenesulfonic acid), etc.

The polypeptides of the present invention may also form a salt with a basic substance. Examples of these basic salts include, for example, salts with inorganic bases such as alkali metal salts (sodium salt, lithium salt, potassium salt, etc.), alkaline earth metal salts, ammonium salts, and the like or salts with organic bases, such as diethanolamine salts, cyclohexylamine salts, and the like.

In one embodiment of the present invention, the various polypeptides of the present invention may be admixed in various combinations and or admixed with other known proteins or peptides, which are known or believed to facilitate an immunological response, thereby providing protection against Coccidioides spp. infection. In an alternative embodiment, the polypeptides of the present invention may be administered separately, i.e., at different time points, from each or from other proteins or peptides, which are known or believed to facilitate an immunological response, thereby providing protection against Coccidioides spp. infection. For example, the peptide of amino acids 1 to 166 of SEQ ID NO:4 can be combined with one or more additional Coccidioides spp. polypeptides or antigens, such as Ag2/Pra, Ure, Gel1, or non-Coccidioides protein antigens or toxoids, such as tetanus toxoid, diphtheria toxoid, cholera toxoid, ovalbumin (OVA), or keyhole limpet haemocyanin (KLH).

The pharmaceutically acceptable carriers which can be used in the present invention include, but are not limited to, an excipient, a stabilizer, a binder, a lubricant, a colorant, a disintegrant, a buffer, an isotonic agent, a preservative, an anesthetic, and the like which are commonly used in a medical field.

Also, the dosage form, such as injectable preparations (solutions, suspensions, emulsions, solids to be dissolved when used, etc.), tablets, capsules, granules, powders, liquids, liposome inclusions, ointments, gels, external powders, sprays, inhalating powders, eye drops, eye ointments, suppositories, pessaries, and the like, can be used appropriately depending on the administration method, and the polypeptides of the present invention can be accordingly formulated. Pharmaceutical formulations are generally known in the art and are described, for example, in Chapter 25.2 of Comprehensive Medicinal Chemistry, Volume 5, Editor Hansch et al, Pergamon Press 1990.

The present invention also provides compositions containing the polypeptides or fragments thereof containing one or more suitable adjuvants commonly used in the field of immunology and medicine to enhance the immune response in a subject. Examples of such adjuvants include monophosphoryl lipid A (MPL), a detoxified derivative of the lipopolysaccharide (LPS) moiety of Salmonella minnesota R595, which has retained immunostimulatory activities and has been shown to promote Th1 responses when co-administered with antigens (see U.S. Pat. No. 4,877,611; Tomai et al., Journal of Biological Response Modifiers. 1987. 6:99-107; Chen et al., Journal of Leukocyte Biology 1991. 49:416-422; Garg & Subbarao. Infection and Immunity. 1992. 60(6):2329-2336; Chase et al., Infection and Immunity.1986. 53(3):711-712; Masihi et al, Journal of Biological Response Modifiers. 1988. 7:535-539; Fitzgerald, Vaccine 1991. 9:265-272; Bennett et al, Journal of Biological Response Modifiers 1988. 7:65-76; Kovach et al., Journal of Experimental Medicine, 1990. 172:77-84; Elliott et al., Journal of Immunology. 1991.10:69-74; Wheeler A. W., Marshall J. S., Ulrich J. T., International Archives of Allergy and Immunology 2001. Oct;126(2):135-9; and Odean et al., Infection and Immunity 1990. 58(2):427-432); MPL derivatives (see U.S. Pat. No. 4,987,237) other general adjuvants (see U.S. Pat. No. 4,877,611); CpG and ISS oligodeoxynucleotides (see U.S. Pat. No. 6,194,388; U.S. Pat. No. 6,207,646; U.S. Pat. No. 6,239,116; U.S. Pat. No. 6,339,068; McCluskie, M. J., and H. L. Davis. Vaccine 2002. 19:413-422; Ronaghy A, Prakken B J, Takabayashi K, Firestein G S, Boyle D, Zvailfler N J, Roord S T, Albani S, Carson D A, Raz E. hnmunostimulatory DNA sequences influence the course of adjuvant arthritis. Journal of Immunology 2002. 168(1):51-6.; Miconnet et al (2002) 168(3) Journal of Immunology pp 1212-1218; Li et al (2001) Vaccine 20(1-2):148-157; Davis (2000) Devopmental Biology 104:165-169; Derek T. O'Hagan, Mary Lee MacKichan, Manmohan Singh, Recent developments in adjuvants for vaccines against infectious diseases, Biomolecular Engineering 18 (3) (2001) pp. 69-85; McCluskie et al (2001) Critical Reviews in Immunology 21(1-3):103-120); trehalose dimycolate (see U.S. Pat. No. 4,579,945); amphipathic and surface active agents, e.g., saponin and derivatives such as QS21 (see U.S. Pat. No. 5,583,112); oligonucleotides (Yamamoto et al, Japanese Journal of Cancer Research, 79:866-873, 1988); detoxified endotoxins (see U.S. Pat. No. 4,866,034); detoxified endotoxins combined with other adjuvants (see U.S. Pat. No. 4,435,386); combinations with QS-21 (see U.S. Pat. No. 6,146,632); combinations of detoxified endotoxins with trehalose dimycolate and endotoxic glycolipids (see U.S. Pat. No. 4,505,899); combinations of detoxified endotoxins with cell wall skeleton (CWS) or CWS and trehalose dimycolate (see U.S. Pat. Nos. 4,436,727, 4,436,728 and 4,505,900); combinations of just CWS and trehalose dimycolate, without detoxified endotoxins (as described in U.S. Pat. No. 4,520,019); chitosan adjuvants (see U.S. Pat. Nos. 5,912,000; 5,965,144; 5,980,912; Seferian, P. G., and Martinez, M. L. Immune stimulating activity of two new chitosan containing adjuvant formulations (2001) Vaccine. 2000. 19(6):661-8). All of the references cited in this paragraph are incorporated herein by reference.

In another embodiment, the antigenic compositions of the present invention can be provided as an adsorbed vaccine or immunostimulatory composition as described in Matheis et al. (Matheis, M., Zott, A., Schwanig, M. The role of the adsorption process for production and control combined adsorbed vaccines. Vaccine. 2000. 20:67-73), which is incorporated herein by reference.

In another embodiment, various adjuvants, even those that are not commonly used in humans, may be employed in animals where, for example, one desires to subsequently obtain activated T cells or to protect valuable or valued animals from infection due to Coccidioides spp.

VI. Administration of Vaccines

As used herein the subject that would benefit from the administration of the polypeptide and or nucleotide vaccines and formulations described herein include any mammal that can benefit from protection against Coccidioides spp. infection. In a preferred embodiment, the subject is a human. In a second embodiment, the subject is a domestic animal, including but not limited to dog, cat, horse, bovine (meaning any sex or variety of cattle) or other such domestic animals.

By polypeptides capable of eliciting an immune response in a subject human, including vaccination, the invention covers any polypeptide, peptide, peptide mimic, or chemical product capable of inducing an immune reaction that results in or augments the subject's ability to mount some level of immune protection inhibiting Coccidioides spp. infection. In one embodiment, the Coccidioides spp. is Coccidioides immitis. In another embodiment, the Coccidioides spp. is Coccidioides posadasii.

As used herein, “inhibit”, “inhibiting” or “inhibition” includes any measurable or reproducible reduction in the infectivity of Coccidioides spp. in the subject patient. “Reduction in infectivity” means the ability of the subject to prevent or limit the spread of Coccidioides spp. fungus in tissues or organs exposed to or infected by said fungus. Furthermore, “amelioration”, “protection”. “prevention” and “treatment” mean any measurable or reproducible reduction, prevention, or removal of any of the symptoms associated with Coccidioides spp. infectivity, and particularly, the prevention, or amelioration of Coccidioides spp. infection and resultant pathology itself.

Optimum doses of polypeptide that elicit an inhibiting response can be determined through experimentation. Typically, one skilled in the art would design such experiments using animal models to test a range of doses that would result in both inhibitory and non-inhibitory responses, allowing for the selection of appropriate doses.

The dosages used in the present invention to provide immunostimulation include from about 0.1 μg to about 500 μg, which includes, 0.5, 1.0, 1.5, 2.0, 5.0, 10, 15, 20, 25, 30, 35, 40, 45, 50, 55, 60, 65, 70, 75, 80, 85, 90, 95, 100, 150, 200, 250, 300, 350, 400, and 450 μg, inclusive of all ranges and subranges there between. Such amount may be administered as a single dosage or may be administered according to a regimen, including subsequent booster doses, whereby it is effective; e.g., the compositions of the present invention can be administered one time or serially over the course of a period of days, weeks, months and or years. The polypeptide compositions of the present invention can be administered by any suitable administration method including, but not limited to, injections (subcutaneous, intramuscular, intracutaneous, intravenous, intraperitoneal), eye dropping, instillation, percutaneous administration, transdermal administration, oral administration, intranasal administration, inhalation, etc.

VII. Other Uses.

Also included within the scope of the present invention are kits suitable for providing one or more of the polypeptides of the invention. For example, in such a kit one vial can comprise the polypeptides of the invention admixed with a pharmaceutically acceptable carrier, either in a aqueous, non-aqueous, or dry state; and a second vial which can carry immunostimulatory agents, and or a suitable diluent for the peptide composition, which will provide the user with the appropriate concentration of peptide to be delivered to the subject. In one embodiment, the kit will contain instructions for using the polypeptide composition and other components; as included, such instructions can be in the form of printed, electronic, visual, and or audio instructions. The vaccinations will normally be at two to twelve week intervals, more usually from three to five week intervals. Periodic boosters at intervals of 1-5 years, usually three years, will be desirable to maintain protective levels. The course of the immunization may be followed by assays for activated T cells produced, skin-test reactivity, or other indicators of an immune response to Coccidioides spp.

The polypeptide of the invention can be used to detect the presence of antibodies in the sera of patients potentially infected with Coccidioides spp. Antibodies that react specifically with the inventive polypeptides can be used to detect the presence of circulating antigens in the sera of patients potentially infected with Coccidioides spp. Such detection systems include radioimmunoassays and various modifications thereof which are well known to those skilled in the art. In addition, the polypeptide of the invention can be used to detect the presence of a cell-mediated immune response in a biological sample. Such assay systems are also well-known to those skilled in the art and generally involve the clonal expansion of a sub-population of T cells or the production of cytokines in response to stimuli from the polypeptide or detection of reactive T cells by flow cytometry or other methods known to those skilled in the art; e.g., methods described by Richards et al. (Richards, J. O., Ampel, N. M., Galgiani, J. N. and Lake, D. F.). When so-used, the humoral and or cell-mediated response of a patient can be determined and monitored over the course of the disease. Methods of generating antibodies directed to a specific peptide fragment are known in the art. Examples of such methods are disclosed in Antibodies, A Laboratory Manual, Harlow and Lane, Cold Spring Harbor Press, 1988, herein incorporated by reference.

Having generally described this invention, a further understanding can be obtained by reference to certain specific examples that are provided herein for purposes of illustration only, and are not intended to be limiting unless otherwise specified.

### EXAMPLES

### Example 1

Cloning, and Expression in E. coli of 1,3-β-glucanosyltransferease Homologue (GEL1) from Coccidioides posadasii

Materials and Methods

Culture conditions and parasitic cell development.

C. posadasii (isolate C735) was used for all experimental procedures reported in this study. The saprobic (mycelial) phase of the fungus was grown on glucose-yeast extract agar (GYE; 1% glucose, 0.5% yeast extract, 2% agar). Arthroconidia (asexual reproductive products of the saprobic phase) were isolated from these plate cultures, suspended in PBS, and used to inoculate either liquid GYE medium or a defined glucose-salts medium for growth of the mycelial or parasitic phases, respectively. In vitro growth of parasitic cells (spherules) of C. posadasii demonstrated near-synchronous development during the first generation of the parasitic cycle. Spherules obtained from cultures at 72 h or 132 h after inoculation with arthroconidia are distinguished by their morphogenetic features; 72 h spherules are in late stage of isotropic growth and early stage of segmentation, while 132 h spherules are mature and the majority (approximately 80%) have ruptured to release their endospores (Cole, G. T., C.-Y. Hung, and N. Delgado. 2002. Parasitic phase-specific gene expression in Coccidioides. ASM News 68:603-611).

Genome database analysis and gene discovery. The C. posadasii genome sequencing project was initiated in 2001 at The Institute for Genomic Research (TIGR, Rockville, Md.), and involves a whole genome shotgun strategy for determination of >99% of the 29-megabase genome sequence. Genomic libraries of C. posadasii (isolate C735) with inserts of 2-10 kilobases (kb) were constructed in the pUC plasmid (Promega, Madison, Wis.), and sequenced from both ends. Each library contained >6×105 recombinants, and the combined recombinants of three libraries have been estimated to be sufficient for sequence analysis of the entire C. posadasii genome (Kirkland, T. N., and G. T. Cole. 2002. Gene-finding in Coccidioides immilis: searching for immunogenic proteins, p. 247-254. In K. J. Shaw (ed.), Pathogen genomics: impact on human health. Humana Press, Totowa, N.J.). Genomic survey sequences (GSS) have been assembled into unique contigs and incorporated into a public database (available at The Institute for Genomic Research web site at tigr.org). Computational analyses of the partial genome database were performed by application of the basic local alignment search tool (BLAST) (Altschul, S. F., T. L. Madden, A. A. Schäffer, J. Zhang, Z. Zhang, W. Miller, and D. J. Lipman. 1997. Gapped BLAST and PSI-BLAST: a new generation of protein database search programs. Nucleic Acids Research 25:3389-3402). Sequence alignments were conducted using the translated nucleotide sequences of the contigs and the non-redundant protein database available from the National Center for Biotechnology Information (Wheeler, D. L., C. Chappey, A. E. Lash, D. D. Leipe, T. L. Madden, G. D. Schuler, T. A. Tatusova, and B. A. Rapp. 2000. Database resources of the National Center for Biotechnology Information. Nucleic Acids Research 28:10-14). BLASTX matches were selected with Expect (E) values of <10⁻⁴ as previously described (Kirkland, T. N., and G. T. Cole. 2002. Gene-finding in Coccidioides immitis: searching for immunogenic proteins, p. 247-254. In K. J. Shaw (ed.), Pathogen genomics: impact on human health. Humana Press, Totowa, N.J.). A 495-base pair (bp) fragment of a single contig was selected on the basis of its high translated sequence homology (E=10⁻⁴⁴) to a reported β-1,3-glucanosyltransferase of Aspergillus fumigatus (Gel1; GenBank accession number AF072700) (Hartland, R. P., T. Fontaine, J. P. Debeaupuis, C. Simenel, M. Delepierre, and J.-P. Latgé. 1996. A novel β-(l-3)-glucanosyltransferase from the cell wall of Aspergillus fumigatus. J. Biol. Chem. 271:26843-26849). Sense and antisense primer sequences were selected and synthesized on the basis of regions of the translated 495-bp fragment that aligned with the reported amino acid sequence of the Gel1 protein of A. fumigatus. These nested primers were employed in a polymerase chain reaction (PCR) with genomic template DNA of C. posadasii isolate C735, and a 391-bp product was amplified. The nucleotide sequences of the sense and antisense primers were 5′-CTCCGGAGGTCTCGTCTAT-3′ (SEQ ID NO:7), and 5′-TAAACCTGCGGCAGCACCCGCGCT-3′ (SEQ ID NO:8), respectively. The amplification conditions included an initial denaturation step at 94° C. for 1 min, followed by 30 cycles of denaturation at 94° C. for 10 sec, annealing at 55° C. for 10 sec, and extension at 72° C. for 1 min. The 391-bp PCR amplicon was ligated into the pCR2.1 TOPO cloning vector following manufacturer's protocol (Invitrogen, Carlsbad, Calif.), and the nucleotide sequence of the insert was determined as reported (Hung, C.-Y., J.-J. Yu, P. F. Lehmann, and G. T. Cole. 2001. Cloning and expression of the gene which encodes a tube precipitin antigen and wall-associated β-glucosidase of Coccidioides immitis. Infection and Immunity 69:2211-2222). This gene fragment of C. posadasii was referred to as GEL1.

GEL1 Sequence Analysis.

The rapid amplification of CDNA ends (RACE) procedure was used to obtain the full-length CDNA sequence of GEL1, resolve the start of the 5′ untranslated region (UTR), and locate the poly(A) addition site of the gene as previously described (Hung et al., 2001). Gene-specific primers were used in combination with a universal primer mix for the SMART RACE procedure, according to the protocol provided by the manufacturer (Clontech, Palo Alto, Calif.). The genome walking method (Siebert, P. D., A. Chenchik, E. E. Kellogg, K. A. Lukyanov, and S. A. Lukyanov. 1995. An improved PCR method for walking in uncloned genomic DNA. Nucleic Acids Res. 23:1087-1088) was employed to obtain the full-length genomic sequence of GEL1 using a GenomeWalker™ Kit (Clontech), as described by the manufacturer. Genome walking is a PCR-based method used for sequencing genomic DNA upstream or downstream of a partial gene sequence. Briefly, four restriction libraries of C. posadasii genomic DNA were generated using DraI, EcoRV, PvuI, and StuI restriction enzymes. Adapters were ligated to the restriction fragments and a two-step, long-distance PCR was performed using the Advantage genomic polymerase mix, as described by the manufacturer (Clontech). The primers used for this first-step PCR amplification were as follows: 5′-AGAATGCGGGGACACAATCTTCTGGAACTG-3′(SEQ ID NO:9) derived from the sequenced 391-bp amplicon described above, and the adapter primer (AP-1)(SEQ ID NO:10) provided by the manufacturer. The second-step amplification was a nested PCR and was performed using the GEL1-specific primer, 5′-ACTTATTATCTCTTGGCT AAATGCGGATGT-3′(SEQ ID NO: 11), and the nested adapter primer-2 (AP-2, SEQ ID NO: 12) provided by the manufacturer. To obtain the full-length genomic sequence of GEL1, PCR was conducted using the following sense and antisense primers: 5′-CTCCGTTTGAACTCTCCTCTCTTC-3′ (SEQ ID NO:13), and 5′-AAGGAGTTGTCGCCATGCTGC-3′ (SEQ ID NO:14). All PCR products obtained by the RACE and genome walking procedures were separated by agarose gel electrophoresis (1.2%), isolated and ligated into pCR2.1 TOPO vector, and subjected to nucleotide sequence analysis as reported (Hung et al., 2001). The NCBI-BLAST and PSI-BLAST programs were used to search for sequences in the GenBank and SWISS-PROT databases with similarities to the translated full-length GEL1 sequence (Altschul, S. F., T. L. Madden, A. A. Schäffer, J. Zhang, Z. Zhang, W. Miller, and D. J. Lipman. 1997. Gapped BLAST and PSI-BLAST: a new generation of protein database search programs. Nucleic Acids Res. 25:3389-3402). The CLUSTAL X program, version 1.8 (Thompson, J. D., T. J. Gibson, F. Plewniak, F. Jeanmougin, and D. G. Higgins. 1997. The CLUSTAL X windows interface: flexible strategies for multiple sequence alignment aided by quality analysis tools. Nucleic Acids Research 24:4876-4882), was used to align the translated GEL1 of C. posadasii with homologous sequences. The PROSITE algorithm was used to identify conserved motifs in the translated polypeptide with homology to reported proteins (Hofmann, K., P. Bucher, L. Falquet, and A. Bairoch. 1999. The PROSITE database, its status in 1999. Nucleic Acids Research 27:215-219), and the PSORT and PSORT II algorithms were used for prediction of cellular localization sites of the GEL1 gene product (Horton, P., and K. Nakai. 1997. Better prediction of protein cellular localization sites with the k nearest neighbors classifier. Intelligent Systems for Mol. Biol. 5:147-152; Nakai, K., and M. Kanehisa. 1992. A knowledge base for predicting protein localization sites in eukaryotic cells. Genomics 14:897-911). The C. posadasii GEL1 nucleotide sequences reported in this paper have been submitted to the GenBank database under accession nos. AF288063 (cDNA) and AF395660 (genomic).

Cloning and Expression of GEL1 by Escherichia coli.

Oligonucleotide primers were designed to amplify a 1.3-kb cDNA fragment of the GEL1 gene that encodes amino acids 19-447, the predicted mature protein. The nucleotide sequences of the sense and antisense primers were 5′-AGCTCATATGGCCTACGCGGGTG-3′ (SEQ ID NO:5) and 5′-AGGTCGACCTTTAAAGCATTAAAC-3′ (SEQ ID NO:6), which contained engineered NdeI and SalI restriction sites, respectively (nucleotides in boldface type). The amplification parameters were as follows: an initial denaturation step at 94° C. for 1 min followed by 30 cycles of denaturation at 94° C. for 10 sec, annealing at 50° C. for 10 sec, and extension at 72° C. for 2 min. The 1.3-kb PCR product was digested with NdeI and SalI, separated by agarose gel electrophoresis (1%), excised, and subcloned into the NdeI/SalI site of pET28b (Novagen, Madison, Wis.) to yield the pET28b-GEL1 plasmid construct. Bacterial transformation and purification of the recombinant polypeptide rGel1 were performed as previously reported (Hung et al., 2001). The purified rGel1 polypeptide was subjected to internal amino acid sequence analysis to confirm its identity. The endotoxin content of the stock solution which contained the recombinant protein (1 mg/ml phosphate-buffered saline [0.1 M, pH 7.4]) was deternined using a Limulus amebocyte lysate kit (QCL-1000; BioWhittaker, Walkersille, Md.) as previously reported (Li, K., J. J. Yu, C. Y. Hung, P. F. Lehmann, and G. T. Cole. 2001. Recombinant urease and urease DNA of Coccidioides immitis elicit an imiunoprotective response against coccidioidomycosis in mice. Infection and Immunity 69:2878-2887). The stock solution contained 1.0-1.5 endotoxin units (5.0-7.5 ng of endotoxin) per μg of protein.

Production of Antiserum Against rGel1 and Immunofluorescence Microscopy

The chromatographically-isolated recombinant protein was used to immunize BALB/c mice (6 wk. old) for production of specific antiserum as reported (18). The antiserum was used for immunolocalizaton of the native antigen in parasitic cells of C. posadasii as described below. Preimmune mouse serum was used as a control. Freshly isolated, endosporulating spherules obtained from parasitic phase cultures (132 h) were reacted with either mouse preimmune or anti-rGel1 serum at a 1:200 dilution in PBS. The intact cells were then washed with PBS and prepared for examination by fluorescence microscopy as previously described (Hung, C.-Y., J.-J. Yu, K. R. Seshan, U. Reichard, and G. T. Cole. 2002. A parasitic phase-specific adhesin of Coccidioides immitis contributes to the virulence of this respiratory fungal pathogen. Infection and Immunity 70:3443-3456).

Results

Identification of the GEL1 Gene in the Genome Database.

A BLASTX search of the partial C. posadasii genome revealed a 495-bp open reading frame (ORF) which encodes a homolog of an A. fumigatus β-1,3-glucanosyltransferase (Hartland et al., 1996; Mouyna, I., R. Hartland, T. Fontaine, M. Diaquin, C. Simenel, M. Delepieree, B. Henrissat, and J.-P. Latgé. 1998. A 1,3-β-glucanosyltransferase isolated from the cell wall of Aspergillus fumigatus is a homologue of the yeast Bg12p. Microbiology 144:3171-3180). High sequence homology (64% identity, 78% similarity) was observed between the translated C. posadasii gene fragment and the reported glucan elongating glucanosyltransferase (GEL1) gene of A. fumigatus. Nested, PCR primers were synthesized on the basis of nucleotide sequences of the C. posadasii genome fragment that translated N-terminal and C-terminal regions aligning with near identity to the A. fumigatus Gel1 sequences. These primers amplified a 391-bp fragment of the GEL1 homolog in the presence of genomic template DNA of C. posadasii. Nucleotide sequence analysis of the 391-bp PCR product confirmed that it was identical to the contig sequence originally identified in the C. posadasii genome database.

Full Length GEL1 Sequence Analysis.

The 5′ and 3′ RACE methods yielded cDNA bands of 1.3 kb and 0.5 kb, respectively. The nucleotide sequences of these bands overlapped with the 495-bp fragment of GEL1, and included the 5′ and 3′ UTR sequences of the gene. Application of the 3′ genome walking method resulted in isolation of three genome fragments of 0.4, 1.5, and 3.0 kb. The 1.5 kb band was selected for genomic sequence analysis and was compared to the cDNA sequences obtained from analysis of the RACE products. The complete genomic sequence of the C. posadasii GEL1 gene was obtained as a 3.0-kb PCR product. The open reading frames of the GEL1 cDNA and genomic sequences were resolved. The genomic sequence included six introns of 68, 54, 63, 52, 54, and 71 bp (SEQ ID NO:1). The translated amino acid sequence predicted a protein of 447 residues with a molecular weight of 48.1 kDa and an isoelectric point (pI) of 5.4 (SEQ ID NO:2).

Production of the Recombinant Polypeptide (rGel1).

To express the GEL1 gene of C. posadasii, the 1.3-kb PCR-generated cDNA was subcloned into pET28b to yield the pET28b-GEL1 construct (SEQ ID NO:3). The GEL1 insert lacked the nucleotide sequence that encodes the signal peptide (i.e., residues 1-18 were deleted from rGel1). The predicted molecular size of the rGel1 polypeptide (SEQ ID NO:4) was 48 kDa, which includes the vector-encoded fusion peptide that contained a His tag at its N-terminus. SDS-PAGE was used to examine the composition of the cell lysates obtained from E. coli stain BL21(DE3), which had been transformed with either the empty vector or plasmid construct and then induced with IPTG (isopropyl-β-D-thiogalactopyranoside). The molecular size (MW) of the recombinant protein (rGel1) correlated with the predicted size. The 48-kDa rGel1 was isolated by nickel-affinity chromatography, digested with Lys-C, separated by high pressure liquid chromatography and subjected to N-terminal sequence analysis as described (Hung et al, 2001). The amino acid sequence obtained (DQKVENFTGY (SEQ ID NO: 17)) was identical to the predicted sequence of the translated GEL1 gene (residues 246-255).

Immunolocalization of the Native Gel1.

The isolated rGel1 polypeptide used to raise specific antiserum in mice, recognized the 48-kDa protein in the bacterial lysate and reacted with a surface component of in vitro-grown endospores of C. posadasii, when examined by fluorescence microscopically as previously described (Hung, et al., 2002). The results indicate that rGel1 and native Gel1 share a similar antigenic profile. In contrast, the application of pre-immune negative control serum did not result in fluorescence on the cell surface.

### Example 2

Immunoprotection Studies with rGel1 Polypeptide

Materials and Methods

Immunization and animal challenge. Immunoprotection experiments were conducted with BALB/c (females, 6 wk. old) or C57BL/6 mice (females, 8 wk. old) supplied by the National Cancer Institute (Bethesda, Md.). Mice were challenged with a lethal dose of C. posadasii arthroconidia either by the intraperitoneal (i.p.) or intranasal (i.n.) route. Intraperitoneal challenge delivers a consistent number of fungal conidia to the animals and, although not the natural route of infection, has been shown to be a useful method to initially evaluate candidate vaccine reagents.

For studies evaluating rGel1 in BALB/c mice, three groups of BALB/c mice (20 per group) were subcutaneously (s.c.) immunized with either a control adjuvant comprised of 50 μl of Incomplete Freund's Adjuvant (IFA) plus 10 μg of CpG (unmethylated CpG dinucleotides present in a synthetic oligodeoxynucleotide (ODN) preparation [Integrated DNA Technologies, Inc., Coralville, IA] for a total volume of 100 μl. The CpG ODN sequence used to immunize mice was TCCATGACGTTCCTGACGTT (SEQ ID NO:15) (CpG motifs are underlined), or with two different amounts of rGel1 antigen (1 μg or 5 μg rGel-1 protein, plus adjuvant), as previously reported (Li et al 2001 Infection and Immunity 69:2878-2887). The mice were boosted by s.c. immunization 14 days later with the same amount of immunogen plus adjuvant. Mice were challenged with 70 viable arthroconidia by the i.p. route 2 weeks after the last immunization. Half of the animals were sacrificed 12 days after challenge to examine residual colony-forming units (CFU) in their lungs and spleen using methods as previously described (Li et al., 2001. Infection and Immunity 69:2878-2887),with the remaining animals evaluated for survival over 40 days post-challenge.

For fungal burden studies evaluating rGel1 in C57BL/6 mice, groups of 33 mice were immunized subcutaneously (s.c.) with rGel1 plus adjuvant using essentially the same protocol as previously described (Li, K., J. J. Yu, C. Y. Hung, P. F. Lehmann, and G. T. Cole. 2001. Recombinant urease and urease DNA of Coccidioides immitis elicit an immunoprotective response against coccidioidomycosis in mice. Infection and Immunity 69:2878-2887). The adjuvant used was IFA plus unmethylated CpG ODN. The oligonucleotides were dissolved in PBS (5 mg/ml) and used as stock solution for the vaccination experiments. The mice were then boosted by s.c. immunization 14 days later with the same amount of immunogen plus adjuvant. The animals were subsequently challenged with 80 viable arthroconidia by the intranasal (i.n.) route 4 weeks after the last immunization. Seven animals in each group were sacrificed at Days 0 (just prior to infection) and 7, and 12 post-infection for the determination of immunoglobulin titers and four or three animals, respectively, for evaluation of cytokines, determined either by real-time PCR of bronchiole alveolar lavage fluid (BALF) or by Laser Capture Microdisection methods described below, and the remaining animals were then sacrificed 14 days after infection to determine the residual colony-forming units (CFU) in the lungs and spleen using methods as previously described (Li et al., 2001). The CFU per organ were expressed on a log scale, and the Mann-Whitney U test was used to compare the median numbers as previously described (Kirkland, T. N., F. Finley, K. Orsborn, and J. Galgiani. 1998. Evaluation of the proline-rich antigen of Coccidioides immitis as a vaccine candidate in mice. Infection and Immunity 66:3519-3522).

For survival studies evaluating rGel1 in C57BL/6 mice, groups of 12 mice each were immunized s.c. with 0.2, 1, or 5 μg of rGel1 plus adjuvant as above and then challenged with 80 viable arthroconidia by the i.n. route. Mice were scored for survival over a 40-day period post-challenge. Survival differences between groups of i.n.-infected mice (12 per group) were analyzed for statistical significance by the Kaplan-Meier method.

Cytokine Production in Immunized and Infected Mice.

The cytokine response of C57BL/6 mice immunized with rGel1 plus adjuvant versus adjuvant alone using the laser capture microdissection (LCM) method of mRNA analysis were assessed in animals immunized and infected i.n. as described above. Thin sections of lung tissue at sites of infection were excised by LCM, subjected to total RNA extraction, and sampled by RT-PCR for quantitation of cytokine expression for panels of Th1 vs Th2 cytokines.

Results

Fungal Burden in rGel1-immunized, Intraperitoneally Challenged Mice.

BALB/c mice from the respective treatment groups were challenged by the i.p. route with a lethal inoculum of C. posadasii and then sacrificed 12 days later to examine residual colony-forming units (CFU) in their lungs and spleen. The results showed that the organism was cleared from lungs of mice immunized with two separate doses of 1 μg of rGel1 plus adjuvant, but only partially cleared from the lungs of animals administered 5 μg of the antigen preparation using the same immunization protocol (see Table 1). The median number of organisms in the lungs of control mice immunized with adjuvant alone was 4.5 (log₁₀) CFU (range, 2.1 to 5.1). The difference between the number of CFU detected in the lungs of control mice and animals immunized with 1 μg of rGel1 was statistically significant (P=0.004). Mice immunized with 5 μg of rGel1, on the other hand, did not show a significant reduction in CFU compared to infected control animals. The spleen homogenates of mice immunized with either 1 μg or 5 μg of rGel1 showed a higher fungal burden and wider range of CFU than the lung homogenates of rGel1-immune mice. Nevertheless, the group of mice immunized with 1 μg of rGel1 showed significant reduction of the fungal burden compared to control mice (P=0.014), and significantly greater clearance of C. posadasii from the spleen than mice immunized with 5 μg of the recombinant protein (P=0.032).

TABLE 1
Evaluation of immunoprotection in BALB/c mice
based on residual fungal
burden at 12 days post-infection (i.p. route)

| | Median CFU (log ₁₀) ± S.D. | |
|---|---|---|
| | (P value vs. CpG adjuvant control) | |
| Group | Lungs | Spleen |
| CpG adjuvant (control) | 4.5 ± 1.80 | 5.39 ± 1.99 |
| rGel1 1 μg + CpG | 2.0 ± 0.23 | 2.0 ± 0.66 |
| | (0.004) | (0.014) |
| rGel1 5 μg + CpG | 2.0 ± 1.57 | 3.43 ± 1.34 |
| | (0.097) | (0.099) |

The remaining animals were held and monitored for survival for a total of 40 day post-infection. The results, presented in Table 2, indicate a high degree of protection conferred by immunization with both doses of rGel1, with slightly greater protection seen in the 1 μg group.

TABLE 2
Survival of recombinant Gel1-immunized BALB/c mice
against i.p. challenge with
C. posadasii

| Group | % Survival | Mean of survival days |
|---|---|---|
| CpG adjuvant (control) | 0 | 13.9 |
| rGel1 1 μg + CpG | 91 | 39.3 |
| rGel1 5 μg + CpG | 67 | 32.2 |

Fungal Burden in rGel1-immunized, Intransally Challenged Mice.

C57BL/6 mice from the respective treatment groups were challenged by the i.n. route with a lethal inoculum of C. posadasii and then sacrificed 14 days later to examine residual colony-forming units (CFU) in their lungs and spleen using methods as previously described (Li et al., 2001). The CFU per organ were expressed on a log scale, and the Mann-Whitney U test was used to compare the median numbers as previously described (Kirkland, T. N., F. Finley, K. Orsbom, and J. Galgiani. 1998. Evaluation of the proline-rich antigen of Coccidioides immitis as a vaccine candidate in mice. Infection and Immunity 66:3519-3522). The results, presented in Table 3, demonstrate that immunization with the rGel1 at 1 μg/ml plus adjuvant was able to reduce the fungal burden in both lungs and spleens by more that 2 log units, in comparison to the control groups, in animals challenged by the intranasal route.

TABLE 3
Evaluation of fungal burden in lungs and spleen of
recombinant Gel1-immunized
C57BL/6 mice at 14 days post-challenge by the i.n. route

| | Median CFU (log ₁₀) ± S.D. | |
|---|---|---|
| | (P value vs. CpG adjuvant control) | |
| Group | Lungs | Spleen |
| PBS control | 6.73 ± 0.94 | 2.54 ± 1.27 |
| | (0.80) | (0.76) |
| CpG adjuvant (control) | 6.43 ± 1.09 | 2.73 ± 1.02 |
| RGel1 5 μg + CpG | 7.11 ± 1.11 | 3.63 ± 1.02 |
| | (0.60) | (0.45) |
| RGel1 1 μg + CpG | 4.28 ± 0.78 | 2.0 ± 0.22 |
| | (0.001) | (0.039) |
| RGel1 0.2 μg + CpG | 7.07 ± 1.25 | 2.54 ± 1.08 |
| | (0.90) | (0.94) |

Survival of rGel1-immunized, Intranasally-challenged Mice.

Control C57BL/6 mice (12 mice per group) immunized with PBS or PBS plus adjuvant and then challenged via the intranasal route with a lethal inoculum of C. posadasii (80 viable arthroconidia) typically began to die at about 13 days post-challenge. None of these infected, control mice survived beyond 25 days. Mice immunized with rGel1 (0.2 μg, 1 μg, or 5 μg), on the other hand, all showed significantly greater survival when compared to the control mice (P values are indicated)(see Table 4). However, based on results of the two representative experiments, there was an apparent trend that mice immunized with 1 μg of rGel1 were better protected than those immunized with

TABLE 4
Percent survival and mean of survival days of
C57BL/6 mice immunized with
recombinant Gel1 and challenged by the i.n. route

| Group | % Survival | Mean of survival days |
|---|---|---|
| PBS (control) | 0 | 17.55 |
| CpG adjuvant (control) | 0 | 16.73 |
| rGel1 5 μg + CpG | 33.3 | 26.25 |
| | (p = 0.00005)* | |
| rGel1 1 μg + CpG | 66.7 | 32.42 |
| | (p = 0.00002) | |
| rGel1 0.2 μg + CpG | 25.0 | 25.0 |
| | (p + 0.002) | |

*Experimental group vs. CpG control

either 0.2 μg or 5 μg of rGel1, although the difference in percent survival between these groups of mice was not statistically significant. The consistently greater number of survivors amongst the group of mice immunized with 1 μg of rGel1 suggested that these animals had mounted a more protective immune response against the lethal intranasal challenge of C. posadasii than mice immunized with either the higher or lower dose of the same immunogen, a finding consistent with the fungal burden data.

Cytokine Production in Immunized and Infected Mice.

C57BL/6 mice that had been immunized with rGel1 plus adjuvant vs. adjuvant alone were used to assess the production of Th1 and Th2-related cytokines in lung tissue or in BALF either just prior to or at intervals after i.n. infection by C. posadasii. The results for lung tissue cytokine expression, presented in Tables 5 and 6, show that the rGel1 (1 μg/dose)-immunized mice are characterized by a pronounces Th1 response on the basis of IFN-γ and IL-12 expression by days 7 or 14 after infection, while the lung tissue of the control mice at infection sites showed a Th2 response.

TABLE 5
Comparison of transcript levels of selected Th1 pathway-related cytokines in
LCM-isolated lung tissue of rGel1-immunized,
i.n.-challenged C57BL/6 mice as determined
by quantitative real time RT-PCR (QRT-PCR)

| | | Immunized | | | |
|---|---|---|---|---|---|
| | | mouse groups | | | |
| | | Fold change in transcript level compared to PBS control | | | |
| | | (geometric mean ± S.D.) | | | |
| Sampling | | (P value vs. CpG adjuvant control) | | | |
| time/cytokine tested | | | CpG | | |
| Days | | | adjuvant | rGe1 5 μg + | rGel1 1 μg + |
| post-challenge | Cytokine | PBS control | control | CpG | CpG |
| 0 | IFN-γ | 1 ± 0 | 2.38 ± 0.92 | 2.43 ± 0.17 | 0.21 ± 0.4 |
| | IL-12 | 1 ± 0 | 1.97 ± 0.49 | 1.82 ± 0.66 | 0.95 ± 0.43 |
| 7 | IFN-γ | 1.46 ± 0.7 | 1.07 ± 0.8 | 1.14 ± 0.68 | 2 ± 0.88 |
| | | (0.387) | | (0.667) | (0.18) |
| | IL-12 | 1.47 ± 0.76 | 2.99 ± 0.64 | 0.51 ± 0.58 | 5.03 ± 0.41 |
| | | (0.087) | | (0.0035) | (0.382) |
| 14 | IFN-γ | 1.38 ± 0.43 | 1.35 ± 0.99 | 1.21 ± 0.79 | 13.18 ± 0.73 |
| | | (0.92) | | (0.571) | (0.011) |
| | IL-12 | 0.27 ± 0.29 | 0.49 ± 0.12 | 1.95 ± 0.22 | 5.31 ± 0.66 |
| | | (0.353) | | (0.244) | (0.0017) |

For cytokine production in BALF, the presence of high concentrations of IFN-γ and IL-12 in BALFs of mice immunized with 1 μg/dose of rGel1 contrasts sharply with the lower concentrations of these same cytokines in control mice at 7 and 14 days post-challenge. Mice immunized with 5 μg/dose of rGel1 produced low amounts of all four cytokines examined in the ELISAs (Tables 7 and 8), but did produce slightly higher concentrations of IL-5 and IL-10 at 14 days post-challenge compared to mice immunized with 1 μg/dose of rGel1 (Table 8). This observation supports our earlier conclusion that the high dose of rGel1 appears to elicit a Th2 response.

TABLE 6
Comparison of transcript levels of selected
Th2 pathway-related cytokines in
LCM-isolated lung tissue of rGel1-immunized,
i.n.-challenged C57BL/6 mice as determined
by quantitative real time RT-PCR (QRT-PCR)

| | | Immunized | | | |
|---|---|---|---|---|---|
| Sampling | | mouse groups | | | |
| time/cytokine | | Fold change in transcript level cf. PBS control | | | |
| tested | | (geometric mean ± S.D.) | | | |
| Days | | (P value vs. CpG adjuvant control) | | | |
| post- | | | CpG | | |
| chal- | Cyto- | | adjuvant | rGel1 5 μg + | rGel1 1 μg + |
| lenge | kine | PBS control | control | CpG | CpG |
| 0 | IL-5 | 1 ± 0 | 3.53 ± 0.86 | 3.29 ± 0.75 | 1 ± 0.4 |
| | IL-10 | 1 ± 0 | 3.09 ± 0.67 | 2.64 ± 0.27 | 0.86 ± 0.7 |
| 7 | IL-5 | 2.04 ± 0.79 | 4.35 ± 0.67 | 1.82 ± 0.92 | 0.38 ± 0.13 |
| | | (0.136) | | (0.064) | (0.0024) |
| | IL-10 | 2.27 ± 1.01 | 9.38 ± 0.66 | 6.36 ± 0.52 | 3.48 ± 0.9 |
| | | (0.038) | | (0.219) | (0.018) |
| 14 | IL-5 | 6.59 ± 0.74 | 4.99 ± 1.46 | 2.35 ± 1.15 | 1.09 ± 0.93 |
| | | (0.184) | | (0.057) | (0.015) |
| | IL-10 | 5.39 ± 0.67 | 8.53 ± 1.19 | 7.52 ± 0.88 | 1.16 ± 0.7 |
| | | (0.138) | | (0.606) | (0.005) |

TABLE 7
Cytokine protein concentrations in bronchoalveolar lavage fluid (BALF) from
recombinant Gel1-immunized C57BL/6 mice at days 0, 7, and 14 post-intranasal challenge
as determined by ELISA

| | | Immunized | | | |
|---|---|---|---|---|---|
| | | mouse groups | | | |
| | | Cytokine concentration (pg/ml) | | | |
| Sampling | | (geometric mean ± S.D.) | | | |
| time/cytokine tested | | (P value vs. CpG adjuvant control) | | | |
| Days post- | | | CpG adjuvant | | |
| challenge | Cytokine | PBS control | control | rGel1 5 μg + CpG | rGel1 1 μg + CpG |
| 0 | IFN-γ | 19.58 ± 4.45 | 72.5 ± 17.42 | 63.53 ± 11.84 | 0 |
| | IL-12 | 130.35 ± 56.28 | 141.58 ± 13.27 | 14.15 ± 23.37 | 153.57 ± 73.11 |
| 7 | IFN-γ | 62.08 ± 32.83 | 221.3 ± 22.5 | 31.72 ± 4.0 | 582.24 ± 57.47 |
| | | (0.017) | | (0.0037) | (0.016) |
| | IL-12 | 276.69 ± 48.5 | 302.51 ± 68.02 | 213.77 ± 46.19 | 477.95 ± 29 |
| | | (0.7369) | | (0.024) | (0.018) |
| 14 | IFN-γ | 60.06 ± 20.69 | 157.21 ± 18.85 | 92.73 ± 19.55 | 921.23 ± 167.27 |
| | | (0.051) | | (0.10) | (0.016) |
| | IL-12 | 147.22 ± 12.12 | 406.97 ± 17.82 | 400.11 ± 50.56 | 915.58 ± 27.44 |
| | | (0.00168) | | (0.838) | (0.0005) |

TABLE 8
Cytokine protein concentrations in bronchoalveolar lavage fluid (BALF) from
recombinant Gel1-immunized C57BL/6 mice at days 0, 7, and 14 post-intranasal challenge
as determined by ELISA

| | | Immunized | | | |
|---|---|---|---|---|---|
| | | mouse groups | | | |
| | | Cytokine concentration (pg/ml) | | | |
| Sampling | | (geometric mean ± S.D.) | | | |
| time/cytokine tested | | (P value vs. CpG adjuvant control) | | | |
| Days post- | | | CpG adjuvant | | |
| challenge | Cytokine | PBS control | control | rGel1 5 μg + CpG | rGel1 1 μg + CpG |
| 0 | IL-5 | 46.18 ± 8.35 | 30.79 ± 6.97 | 58.22 ± 4.61 | 4.01 ± 3.59 |
| | IL-10 | 39.85 ± 16.26 | 2.09 ± 3.62 | 26.89 ± 15.79 | 0 ± 0 |
| 7 | IL-5 | 100.04 ± 11.52 | 254.33 ± 47.58 | 61.31 ± 4.18 | 68.75 ± 10.61 |
| | | (0.0187) | | (0.020) | (0.0307) |
| | IL-10 | 83.27 ± 15.37 | 263.44 ± 19.87 | 47.35 ± 12.16 | 31.35 ± 1.4 |
| | | (0.0012) | | (0.0072) | (0.0025) |
| 14 | IL-5 | 463.93 ± 12.54 | 668.51 ± 37.04 | 132.23 ± 7.34 | 78.07 ± 7.45 |
| | | (0.0107) | | (0.0015) | (0.0019) |
| | IL-10 | 225.05 ± 26.19 | 622.56 ± 29.73 | 140.35 ± 21.34 | 26.08 ± 9.88 |
| | | (0.00004) | | (0.0008) | (0.0009) |

Results for specific immunoglobulin titers to rGel1 immunized at a dose 1 μg is consistent with a stimulation a Th1 pathway of immune response (Table 9). The IgG2a antibody titers to rGel1 showed a sharp increase from day 0 and remained high between days 7 and 14 post-challenge, as detected by ELISAs with sera from mice immunized with 1 μg/dose of rGel1. The IgG1 antibody titers in mice immunized with 5 μg/dose of rGel1 sharply increased between day 0 and day 7 post-challenge and then decreased at day 14. These data suggest that mice immunized with the lower dose of rGel1 establish and maintain a Th1 immune response to infection, while mice immunized with 5 μg/dose of the same antigen fail to mount a Th1 response to C. posadasii infection and instead maintain a Th2 response.

Thus, the data derived from survival and fungal load studies, combined with the results from the immunological assays, demonstrate the efficacy of Gel1 as a vaccine for coccidioidomycosis.

TABLE 9
Determination of titers of recombinant Gel1-specific immunoglobulin in sera of
immunized C57BL/6 mice at 0, 7, and 14 days post-intranasal challenge

| | | Immunoglobulin titers in sera of mice | | | |
|---|---|---|---|---|---|
| Sampling | Ig tested | subjected to different immunization protocols | | | |
| time | Immuno- | (geometric mean ± S.D.) | | | |
| Days post- | Globulin | | CpG adjuvant | | |
| challenge | (Ig) | PBS control | control | rGel1 5 μg + CpG | rGel1 1 μg + CpG |
| 0 | Total | 211.25 ± | 210.52 ± 429.04 | 30924.8 ± 1423.77 | 44021.37 ± 2087.54 |
| | IgG | 169.82 | | | |
| | IgG1 | 201.45 ± 32.56 | 253.23 ± 58.74 | 22301.59 ± 2439.83 | 19751.67 ± 2410.54 |
| | IgG2a | 168.92 ± 54.71 | 205.0 ± 89.32 | 6269.63 ± 2657.83 | 33384.59 ± 9102.45 |
| 7 | Total | 192.92 ± | 108.49 ± 76.03 | 104956.1 ± 8843.43 | 57003.24 ± 1019.95 |
| | IgG | 141.22 | | | |
| | IgG1 | 201.45 ± 32.56 | 178.98 ± 59.31 | 71327.54 ± 10557.88 | 4346.31 ± 2839.09 |
| | | | | *(0.0069) | **(0.012) |
| | | | | **(0.01) | |
| | IgG2a | 200.13 ± 31.23 | 176.29 ± 45.07 | 8785.1 ± 474.1 | 54116.65 ± 3283.86 |
| | | | | *(0.0127) | |
| 14 | Total | 237.07 ± 79.31 | 175.19 ± 52.43 | 87779.44 ± 22571.3 | 31879.61 ± 1127.24 |
| | IgG | | | | |
| | IgG1 | 212.45 ± 32.66 | 160.22 ± 50.14 | 46488.78 ± 2934.68 | 15826.65 ± 1368.42 |
| | | | | *(0.0057) | **(0.0077) |
| | | | | **(0.0121) | |
| | IgG2a | 198.02 ± 30.15 | 173.26 ± 46.32 | 28681.57 ± 2974.95 | 54116.65 ± 8383.16 |
| | | | | *(0.01) | |

P value [*] = 5 μg rGel1 vs. 1 μg rGel1 mouse group; [**] = IgG1 vs. IgG2a in same mouse group

### Example 3

A C. posadasii rGel1 Construct for Expression in Saccharomyces cerevisiae

Oligonucleotides were designed to amplify a 1.3 kb CDNA fragment of the C. posadasii GEL1 gene. Restriction sites for EcoRI and SalI enzymes were incorporated in the forward and reverse primers, respectively. The PCR product was cloned into the vector pGEM-T (Promega Corporation, Madison, Wis., USA) by the T/A-cloning method, and then used to transform competent E. coli cells (TAM-1) via standard heat-shock at 42° C. Transformants were selected from Luria Bertani (LB) agar plates with 50 μg/ml ampicillin, cultured overnight in LB medium at 37° C. in a shaking incubator, and subsequently PCR screened for presence of the GEL1 insert using the aforementioned primers. PCR-positive clones were then purified using the QIAprep Spin Miniprep Kit (Qiagen, Valencia, Calif., USA). Multiple clones were sequenced using an ABI PRISM 310 Genetic Analyzer (Perkin Elmer, Norwalk, Conn., USA), and the nucleotide sequences were examined to verify the quality of the GEL1 insert. The pGEM-T vector with the GEL1 insert and the YEpFLAG-1 Yeast Expression Vector (Sigma-Aldrich Biotechnology, St. Louis, Mo., USA) were then simultaneously restricted with EcoRI and SalI. Restricted pGEM-T/GEL1 and YEpFLAG-1 vectors were electrophoresed in a 1.2% agarose gel alongside DNA molecular weight markers, sliced from the agarose using a scalpel, purified by gel extraction (Qiagen), and ligated using the Fast-Link DNA Ligation Kit (Epicentre, Madison, Wis., USA) to yield the YEpFLAG-1-GEL1 plasmid construct. The YEpFLAG-1 vector containing the 1.3 kb GEL1 CDNA insert was then used to transform competent E. coli cells (TAM-1) via standard heat-shock at 42° C. Transformants were again selected, cultured overnight in LB medium at 37° C., and PCR screened with the aforementioned primers. PCR-positive clones were then purified using the QIAprep Spin Miniprep Kit (Qiagen). Plasmid sequencing was conducted, as described above, to verify the quality of the GEL1 insert in the YEpFLAG-1 vector. Purified plasmid was then used to transform Saccharomyces cerevisiae (strain BJ3505, Sigma-Aldrich). The EasyComp Transformation Kit reagents (Invitrogen, Carlsbad, Calif., USA) were used to transform S. cerevisiae. The entire transformation was then plated on Synthetic Complete Medium (SCM: 0.1% Yeast Synthetic Drop-out Supplement without Tryptophan, 0.67% Yeast Nitrogen Base without amino acids, 2% glucose) plates minus tryptophan and incubated for three days at 30° C. to allow for selection. Clones that grew in the absence of tryptophan were chosen from the plates and cultured in 50 ml of liquid SCM minus tryptophan at 30° C., for twenty-four hours, in a shaking incubator. The SCM minus tryptophan starter cultures were then used to inoculate Yeast Peptone High Stability Expression Media (YPHSEM: 1% yeast extract, 8% peptone, 1% glucose, 3% glycerol, 20 mM CaCl₂) at a ratio of approximately one-to-ten. These cultures were incubated at 30° C. for four to five days in a shaking incubator to allow for expression of the recombinant protein. Yeast culture supernatant containing the recombinant protein was isolated by centrifugation at 4,000×g for 10 min. The culture supernatant was then concentrated via dialysis in BioDesign Dialysis Tubing with an 8,000 Da Molecular Weight Cut Off (BioDesign Inc., Carmel, N.Y., USA) saturated with Polyethylene Glycol 8000 (Fisher Biotech, Fair Lawn, N.J., USA). The concentrated supernatant was then run on an Anti-FLAG M1 Affinity Gel Column (Sigma-Aldrich) to allow for purification and recovery of the recombinant protein fused to the FLAG peptide.

Obviously, numerous modifications and variations on the present invention are possible in light of the above teachings. It is therefore to be understood that within the scope of the appended claims, the invention may be practiced otherwise than as specifically described herein.

What is claimed is:

1. An isolated nucleic acid selected from the group consisting of: a nucleic acid comprising the sequence of nucleotides 327 to 2032 of SEQ ID NO:1, a nucleic acid encoding the polypeptide comprised of amino acids 1 to 447 of SEQ ID NO:2, a nucleic acid encoding the polypeptide comprised of amino acids 19 to 447 of SEQ ID NO:2 and a nucleic acid encoding the polypeptide comprised of amino acids 19 to 447 of SEQ ID NO:2 with conservative amino acid substitutions.

2. An expression vector comprising the nucleic acid of claim 1.

3. The expression vector of claim 2, further comprising a recombinant regulatory sequence operably linked to said nucleic acid.

4. The expression vector of claim 3, wherein said regulatory sequence is a promoter.

5. The expression vector of claim 3, wherein said regulatory sequence comprises one or more transcriptional regulatory elements that control expression of the nucleic acid in a host cell.

6. The expression vector of claim 5, wherein said host cell is selected from the group consisting of yeast, plant, animal, human and bacterial cells.

7. A host cell comprising the expression vector of claim 2.

8. The host cell of claim 7, wherein said host cell is selected from the group consisting of yeast, plant, animal, human and bacterial cells.

9. The nucleic acid of claim 1, further comprising at least a second coding region that encodes a second, distinct Coccidioides spp. protein, polypeptide or peptide.

10. An isolated nucleic acid selected from the group consisting of: a nucleic acid comprised of nucleotides 64 to 1353 of SEQ ID NO:3, a nucleic acid encoding the polypeptide comprised of amino acids 22 to 450 of SEQ ID NO:4 and a nucleic acid encoding the polypeptide comprised of amino acids 22 to 450 of SEQ ID NO:4 with conservative amino acid substitutions.

11. An expression vector comprising the nucleic acid of claim 10.

12. The expression vector of claim 11, further comprising a recombinant regulatory sequence operably linked to said nucleic acid.

13. The expression vector of claim 12, wherein said regulatory sequence is a promoter.

14. The expression vector of claim 12, wherein said regulatory sequence comprises one or more transcriptional regulatory elements that control expression of the nucleic acid in a host cell.

15. The expression vector of claim 14, wherein said host cell is selected from the group consisting of yeast, plant, animal, human and bacterial cells.

16. A host cell comprising the expression vector of claim 11.

17. The host cell of claim 16, wherein said host cell is selected from the group consisting of yeast, plant, animal, human and bacterial cells.

18. The nucleic acid of claim 10, further comprising at least a second coding region that encodes a second, distinct Coccidioides spp. protein, polypeptide or peptide.

19. An isolated polypeptide selected from the group consisting of: a polypeptide encoded by the nucleotide sequence of nucleotides 327 to 2032 of SEQ ID NO:1, a polypeptide comprised of the amino acid sequence of amino acids 1 to 447 of SEQ ID NO:2, a polypeptide comprised of the amino acid sequence of amino acids 19 to 447 of SEQ ID NO:2 and a polypeptide comprised of the amino acid sequence of amino acids 19 to 447 of SEQ ID NO:2 with conservative amino acid substitutions.

20. A composition comprising the polypeptide of claim 19.

21. The composition of claim 20, wherein said composition includes a pharmaceutically acceptable carrier.

22. The composition of claim 20, further including an adjuvant.

23. The composition of claim 20, further comprising at least a second Coccidioides spp. polypeptide.

24. The composition of claim 23, wherein said second Coccidioides spp. polypeptide is Ag2/PRA1-106.

25. A method of eliciting an immune response in a mammal, comprising introducing into the mammal the composition claim 20, in an amount sufficient to elicit an immune response.

26. The method of claim 25 wherein said mammal is a human.

27. The method of claim 25 wherein said mammal is a domestic animal selected from the group consisting of dog, cat, horse, and bovine.

28. A method of generating antibodies specific for Gel1, comprising introducing into the mammal the composition of claim 20, in an amount sufficient to elicit an antibody response.

29. An isolated polypeptide selected from the group consisting of: a polypeptide encoded by the nucleotide sequence of nucleotides 64 to 1353 of SEQ ID NO:3, a polypeptide comprising the amino acid sequence of amino acids 22 to 450 of SEQ ID NO:4, a polypeptide comprised of the amino acid sequence of amino acids 22 to 450 of SEQ ID NO:4 with conservative amino acid substitutions and a polypeptide consisting of amino acids 22 to 450 of SEQ ID NO:4.

30. A composition comprising the polypeptide of claim 29.

31. The composition of claim 30, wherein said composition includes a pharmaceutically acceptable carrier.

32. The composition of claim 30, further including an adjuvant.

33. The composition of claim 30, further comprising at least a second Coccidioides spp. polypeptide.

34. The composition of claim 33, wherein said second Coccidioides spp. polypeptide is Ag2/PRA1-106.

35. A method of eliciting an immune response in a mammal, comprising introducing into the mammal the composition of claim 30, in an amount sufficient to elicit an immune response.

36. The method of claim 35 wherein said mammal is a human.

37. The method of claim 35 wherein said mammal is a domestic animal selected from the group consisting of dog, cat, horse, and bovine.

38. A method of generating antibodies specific for rGel1 comprising introducing into a mammal the composition of claim 30, in an amount sufficient to elicit an antibody response.

39. The method of claim 38, wherein said mammal is a human.

40. The method of claim 38, wherein said mammal is a domestic animal selected from the group consisting of dog, cat, horse, and bovine.

41. The method of claim 38, wherein said composition further includes a pharmaceutically acceptable carrier.

42. The method of claim 38, wherein said composition further includes an adjuvant.

43. A vector that (a) infects a human cell and (b) is stably transformed with and expresses a heterologous DNA comprising the nucleotide sequence of nucleotides 64 to 1353 of SEQ ID NO:3.

44. The vector of claim 43 wherein said vector encodes a polypeptide, the amino acid sequence of which consists of amino acids 22 to 450 of SEQ ID NO:4.

45. A method of vaccinating a mammal from coccidioidomycosis, comprising: administering the composition of claim 30 in an amount sufficient to elicit an immune response wherein said immune response is sufficient to suppress or attenuate growth of Coccidioides spp. fungus in a mammal infected with said fungus.

46. The method of claim 45 wherein said mammal is a human.

47. The method of claim 45 wherein said mammal is a domestic animal selected from the group consisting of dog, cat, horse, and bovine.

48. The method of claim 45 wherein said composition further includes a pharmaceutically acceptable carrier.

49. The method of claim 45, wherein said composition further includes an adjuvant.

50. A kit, comprising the isolated polypeptide of claim 29.

51. The kit of claim 50, further including an adjuvant.

52. The kit of claim 50, further including instructions for use.


SEQUENCE LISTING (17 sequences)

SEQ ID NO: 1 - DNA, 2972 bp, Coccidioides posadasii
ctccgtttgaactctcctctcttcgtcaaaccgcttctcttccttgtctctcgctccatccccgttctcttcgtcaattctcaaggcaggaagggacctgaggctgccattcttttcagtttggcccttccgccgctgctcgctcgctgctctaccttcttcgtcaatatacaattttaattcatcgccttcgttgatcatccatccgttgtttaccgctgctttcttgtaaatctcccttctgcattcaagtcttccttcatcaacagcttgttcgatttaagcgcttatattgctgcggaactcgaccccatccatctccaaccatgaaaccttcatccgtttccttggtggcggccgccgccatgggagctacatccgcctacgcgggtgtggtgacagctagaggctccaagacacctgccatcaccatcaaaggcaacggtatgttgttgcggttttcgcgccatcttctagcacgttaactaactgaaaacgttttcccttgctagccttcttccaaggcgatgagcgattctatattcgtggactgtatggctgaaccccgactcctttctgcgcgacctgatactgactgcctaacagtgactatcagccggttggtggcaaacaactcaatatatctcctaatgtaactaaaggtctaacctcccacatgtagggtggttcgtcaaaggttgaggacccaattgctgaaacagaaacctgcaagcgggacatcgaatacttcaaggaattgggtgtcaacacaatccgaatctatactgtcgataactccaaaaaccacgatgagtgcatgaaggctctctcagatgctgggatttatctcgttcttgatgtcaacactcccaagtactcgatcaatcgcaaaggtgggagtgacacctccttcggtatctggagccaaattctaactccatccagaacccaaggcgtcctacaacgacgtctatctccagaatgcctttgcaactgtcgaaatgtttgccaaatatgataacactctggtgagatgaagatgtaaagccccgtggcccgttgcttcgactaatctcgctcaggcattcttctctggaaatgaagttatcaacgatggcgaaacttcgaacaccgctccttacgttaaggccgtgactcgtgacattcgtcaattcattcgagcccgtggattgagaaaggttccagttgggtactctgccgtgcgtctcacttgattctttcctgctggtcaaccttggaattttggtttttgtgctgacctcattcacaggccgatatcgatacaaaccgtcttgaaatggcacaatatatgaactgtggcactgatgacgagagaagcgatttcttcgctttcaacgactattcgtggtgctctccatcgtctttcaccacttctggatgggatcaaaaggtcgagaatttcactggctacggtttacctcttttcctctcagaatacggttgcaacaccaacaagcgggactggggtgaagtcaaggccttgtactctgataaaatgacacctgtgtactccggaggtctcgtctatgagtattctcaggaacccagcaactatggcttggtccagcttggcaaaggcaaaccaaaggaactcgacgacttcaaagcgttggcaaaggcgttcaagggcacgaagaatccttcaggcgatggaggttacaactcaaccggcggtgccaacccttgtcccaagaagaatgctcccaactgggacgttgacagcgagtctcttcccgctatccccgaacctgcgaagaagtttatgaaagacggccccggcgagggaccaggtctctccggcaaaggcagccagaatgcggggacacaatcttctggaactgccacgcctggatctggaagcgttgatcctacatccagcgcgggtgctgccgcaggtttacggcctgagtttggtattgctcctatgatgtgcgctatgctggtcgtcttgtcaagcatgtttggagcttcgtttatctttatggtttaatgctttaaacgtgtaccggtctatcgtgtggattcgggtggtcgtgggctcgtacatgcaattctggcgctcttaagttttcactgattctctttctcaaagccagcccggtttgttttcttctggaaatgtattttaattcataggtgccgtgctggattaatcgatccttgaatcagcctcaatttgcatcctcactacttattatctcttggctaaatgcggatgtctcacgagagcggcttcgctcctccgatattccaacctcctccggtggccagtttgtaattatcaagggggtttgctacaaatgttatgccaccccccaataactgtgggttattccggcacccctcctcaaacaaccatcccaaaatagcctatagttagttatatgaggaaatggagttttaaagtattcataaatcaggctcattctctattggattaccttcaagcgcggaagttgactgacctacaaaggctatggtcgaagcaactcctccccaaggatctaaacacctcagagatgtcactgaaggattttctacttttaaagttggaggatattcgtcttctggtgttggaccatgggctaaacattgtcgatgtgccttttgagtatatcattgaagcatgctaggtatgatccggtgtcgagattctgttggaaacggaaatatcttatttgaaggggggaaggcatagtattttggtaaaactataatgtattgcaagactattttaaagaatgagatactcgagagacaagatgggaatataaagaaacattatcgaggtggaaaaaagatggcttcctggatacacgcgagtgggttgatttgcgtgttggaaaatggcagcttctcatatagcttcccacagcagtacttttctcatccccgctgatccggtcccagcagcatggcgacaactcctt

SEQ ID NO: 2 - protein, 447 aa, Coccidioides posadasii
MKPSSVSLVAAAAMGATSAYAGVVTARGSKTPAITIKGNAFFQGDERFYIRGLDYQPGGSSKVEDPIAETETCKRDIEYFKELGVNTIRIYTVDNSKNHDECMKALSDAGIYLVLDVNTPKYSINRKEPKASYNDVYLQNAFATVEMFAKYDNTLAFFSGNEVINDGETSNTAPYVKAVTRDIRQFIRARGLRKVPVGYSAADIDTNRLEMAQYMNCGTDDERSDFFAFNDYSWCSPSSFTTSGWDQKVENFTGYGLPLFLSEYGCNTNKRDWGEVKALYSDKMTPVYSGGLVYEYSQEPSNYGLVQLGKGKPKELDDFKALAKAFKGTKNPSGDGGYNSTGGANPCPKKNAPNWDVDSESLPAIPEPAKKFMKDGPGEGPGLSGKGSQNAGTQSSGTATPGSGSVDPTSSAGAAAGLRPEFGIAPMMCAMLVVLSSMFGASFIFMV

SEQ ID NO: 3 - DNA, 1353 bp, Artificial Sequence
  Recombinant construct
atgggcagcagccatcatcatcatcatcacagcagcggcctggtgccgcgcggcagccatatggcctacgcgggtgtggtgacagctagaggctccaagacacctgccatcaccatcaaaggcaacgccttcttccaaggcgatgagcgattctatattcgtggacttgactatcagccgggtggttcgtcaaaggttgaggacccaattgctgaaacagaaacctgcaagcgggacatcgaatacttcaaggaattgggtgtcaacacaatccgaatctatactgtcgataactccaaaaaccacgatgagtgcatgaaggctctctcagatgctgggatttatctcgttcttgatgtcaacactcccaagtactcgatcaatcgcaaagaacccaaggcgtcctacaacgacgtctatctccagaatgcctttgcaactgtcggaatgtttgccaaatatgataacactccggcattcttctctggaaatgaagttatcaacgatggcgaaacttcgaacaccgctccttacgttaaggccgtgactcgtgacattcgtcaattcattcgagcccgtggattgagaaaggttccagttgggtactctgccgccgatatcgatacaaaccgtcttgaaatggcacaatatatgaactgtggcactgatgacgagagaagcgatttcttcgctttcaacgactattcgtggtgctctccatcgtctttcaccacttctggatgggatcaaaaggtcgagaatttcactggctacggtttacctcttttcctctcagaatacggttgcaacaccaacaagcgggactggggtgaagtcaaggccttgtactctgataaaatgacacctgtgtactccggaggtctcgtctatgagtattctcaggaacccagcaactatggcttggtccagcttggcaaaggcaaaccaaaggaactcgacgacttcaaagcgttggcaaaggcgttcaagggcacgaagaatccttcaggcgatggaggttacaactcaaccggcggtgccaacccttgtcccaagaagaatgctcccaactgggacgttgacagcgagtctcttcccgctatccccgaacctgcgaagaagtttatgaaagacggccccggcgagggaccaggtctctccggcaaaggcagccagaatgcggggacacaatcttctggaactgccacgcctggatctggaagcgttgatcctacatccagcgcgggtgctgccgcaggtttacggcctgagtttggcattgctcctatgatgtgcgctatgctggtcgtcttgtcaagcatgtttggagcttcgtttatctttatggtttaa

SEQ ID NO: 4 - protein, 450 aa, Artificial Sequence
  Fusion peptide
MGSSHHHHHHSSGLVPRGSHMAYAGVVTARGSKTPAITIKGNAFFQGDERFYIRGLDYQPGGSSKVEDPIAETETCKRDIEYFKELGVNTIRIYTVDNSKNHDECMKALSDAGIYLVLDVNTPKYSINRKEPKASYNDVYLQNAFATVGMFAKYDNTPAFFSGNEVINDGETSNTAPYVKAVTRDIRQFIRARGLRKVPVGYSAADIDTNRLEMAQYMNCGTDDERSDFFAFNDYSWCSPSSFTTSGWDQKVENFTGYGLPLFLSEYGCNTNKRDWGEVKALYSDKMTPVYSGGLVYEYSQEPSNYGLVQLGKGKPKELDDFKALAKAFKGTKNPSGDGGYNSTGGANPCPKKNAPNWDVDSESLPAIPEPAKKFMKDGPGEGPGLSGKGSQNAGTQSSGTATPGSGSVDPTSSAGAAAGLRPEFGIAPMMCAMLVVLSSMFGASFIFMV

SEQ ID NO: 5 - DNA, 23 bp, Artificial Sequence
  Primer
agctcatatggcctacgcgggtg

SEQ ID NO: 6 - DNA, 24 bp, Artificial Sequence
  Primer
aggtcgacctttaaagcattaaac

SEQ ID NO: 7 - DNA, 19 bp, Artificial Sequence
  Primer
ctccggaggtctcgtctat

SEQ ID NO: 8 - DNA, 24 bp, Artificial Sequence
  Primer
taaacctgcggcagcacccgcgct

SEQ ID NO: 9 - DNA, 30 bp, Artificial Sequence
  Primer
agaatgcggggacacaatcttctggaactg

SEQ ID NO: 10 - DNA, 22 bp, Artificial Sequence
  Primer
gtaatacgactcactatagggc

SEQ ID NO: 11 - DNA, 30 bp, Artificial Sequence
  Primer
acttattatctcttggctaaatgcggatgt

SEQ ID NO: 12 - DNA, 19 bp, Artificial Sequence
  Primer
actatagggcacgcgtggt

SEQ ID NO: 13 - DNA, 24 bp, Artificial Sequence
  Primer
ctccgtttgaactctcctctcttc

SEQ ID NO: 14 - DNA, 21 bp, Artificial Sequence
  Primer
aaggagttgtcgccatgctgc

SEQ ID NO: 15 - DNA, 20 bp, Artificial Sequence
  Synthetic peptide
tccatgacgttcctgacgtt

SEQ ID NO: 16 - protein, 21 aa, Artificial Sequence
  Fusion peptide
MGSSHHHHHHSSGLVPRGSHM

SEQ ID NO: 17 - protein, 10 aa, Artificial Sequence
  Synthetic peptide
DQKVENFTGY